(19) United States
(12) Patent Application Publication
(10) Pub. No.: US 2021/0008076 A1
(43) Pub. Date: Jan. 14, 2021

(54) NANOPARTICLE COMPOSITIONS

(71) Applicant: January Therapeutics, Inc., San Diego, CA (US)

(72) Inventors: Raj RAHEJA, San Diego, CA (US); Robin M. JACKMAN, San Diego, CA (US); Jason A. KAHANA, San Diego, CA (US)

(21) Appl. No.: 16/980,697

(22) PCT Filed: Mar. 15, 2019
(86) PCT No.: PCT/US2019/022560

§ 371 Date: Sep. 14, 2020

Related U.S. Application Data
(60) Provisional application No. 62/703,805, filed on Jul. 26, 2018.
(60) Provisional application No. 62/644,223, filed on Mar. 16, 2018.

(51) Int. Cl. A61K 31/5377; A61K 9/16; A61K 31/496; A61K 31/495; A61K 31/4725; A61K 31/519; A61K 31/553; A61K 31/4155

(52) U.S. Cl. CPC A61K 31/5377; A61K 9/1658; A61K 31/496; A61K 31/4155; A61K 31/4725; A61K 31/519; A61K 31/553; A61K 31/495

(57) ABSTRACT

Provided herein are nanoparticle compositions comprising modulators of the Bcl-2 family proteins, and pharmaceutically acceptable carriers.

### CROSS-REFERENCE

This application claims benefit of U.S. Provisional Application No. 62/644,223, filed on Mar. 16, 2018, and U.S. Provisional Application No. 62/703,805, filed on Jul. 26, 2018, both of which are herein incorporated by reference in their entirety.

### BACKGROUND

The B cell lymphoma 2 (Bcl-2) family of proteins regulate apoptotic cell death and the overexpression of Bcl-2 family has been implicated in different malignancies.

### BRIEF SUMMARY OF THE DISCLOSURE

This disclosure provides, for example, nanoparticle compositions comprising compounds that modulate the B cell lymphoma (Bcl-2) family proteins, their use as medicinal agents, and processes for their preparation. The disclosure also provides for the use of the nanoparticle compositions described herein as medicaments and/or in the manufacture of medicaments for the treatment of a variety of diseases, including cancer.

Provided in one aspect is a composition comprising nanoparticles, wherein the nanoparticles comprise a modulator of a Bcl-2 family protein; and a pharmaceutically acceptable carrier; wherein the pharmaceutically acceptable carrier comprises albumin.

In some embodiments, the modulator of the Bcl-2 family protein is a modulator of Bcl-2, Bcl-xL, Bcl-w, Bcl-b, A1, and/or Mcl-1. In some embodiments, the modulator of the Bcl-2 family protein is a modulator of Bcl-2, Bcl-xL, Bcl-w, and/or Mcl-1. In some embodiments, the modulator of the Bcl-2 family protein is a modulator of Bcl-2, Bcl-xL, and/or Mcl-1.

In some embodiments, the nanoparticles have an average diameter of about 1000 nm or less for at least about 15 minutes after nanoparticle formation. In some embodiments, the nanoparticles have an average diameter of about 10 nm or greater for at least about 15 minutes after nanoparticle formation. In some embodiments, the nanoparticles have an average diameter of from about 10 nm to about 1000 nm for at least about 15 minutes after nanoparticle formation.

In some embodiments, the nanoparticles have an average diameter of about 1000 nm or less for at least about 2 hours after nanoparticle formation. In some embodiments, the nanoparticles have an average diameter of about 10 nm or greater for at least about 2 hours nanoparticle formation. In some embodiments, the nanoparticles have an average diameter of from about 10 nm to about 1000 nm for at least about 2 hours after nanoparticle formation.

In some embodiments, the nanoparticles have an average diameter of from about 10 nm to about 1000 nm. In some embodiments, the nanoparticles have an average diameter of from about 30 nm to about 250 nm. In some embodiments, the nanoparticles have an average diameter of from about 50 nm to about 200 nm. In some embodiments, the nanoparticles have an average diameter of from about 50 nm to about 150 nm. In some embodiments, the nanoparticles have an average diameter of from about 50 nm to about 100 nm.

In some embodiments, the albumin is human serum albumin. In some embodiments, the molar ratio of the modulator to pharmaceutically acceptable carrier is from about 1:1 to about 20:1. In some embodiments, the molar ratio of the modulator to pharmaceutically acceptable carrier is from about 2:1 to about 12:1. In some embodiments, the nanoparticles are suspended, dissolved, or emulsified in a liquid. In some embodiments, the composition is sterile filterable.

In some embodiments, the composition is dehydrated. In some embodiments, the composition is a lyophilized composition. In some embodiments, the composition comprises from about 0.9% to about 24% by weight of the modulator. In some embodiments, the composition comprises from about 1.8% to about 16% by weight of the modulator. In some embodiments, the composition comprises from about 76% to about 99% by weight of the pharmaceutically acceptable carrier. In some embodiments, the composition comprises from about 84% to about 98% by weight of the pharmaceutically acceptable carrier.

In some embodiments, the composition is reconstituted with an appropriate biocompatible liquid to provide a reconstituted composition. In some embodiments, the appropriate biocompatible liquid is a buffered solution. In some embodiments, the appropriate biocompatible liquid is a solution comprising dextrose. In some embodiments, the appropriate biocompatible liquid is a solution comprising one or more salts. In some embodiments, the appropriate biocompatible liquid is sterile water, saline, phosphate-buffered saline, 5% dextrose in water solution, Ringer's solution, or Ringer's lactate solution. In some embodiments, the nanoparticles have an average diameter of from about 10 nm to about 1000 nm after reconstitution. In some embodiments, the nanoparticles have an average diameter of from about 30 nm to about 250 nm after reconstitution. In some embodiments, the nanoparticles have an average diameter of from about 50 nm to about 200 nm after reconstitution. In some embodiments, the nanoparticles have an average diameter of from about 50 nm to about 150 nm after reconstitution. In some embodiments, the nanoparticles have an average diameter of from about 50 nm to about 100 nm after reconstitution.

In some embodiments, the modulator is a modulator of Bcl-2. In some embodiments, the modulator is a modulator of Bcl-xL. In some embodiments, the modulator is a modulator of Bcl-w. In some embodiments, the modulator is a modulator of Mcl-1. In some embodiments, the modulator is a modulator of Bcl-2 and Bcl-xL. In some embodiments, the modulator is a modulator of Bcl-2, Bcl-xL, and Bcl-w. In some embodiments, the modulator is a modulator of Bcl-2, Bcl-xL, Bcl-w, and Mcl-1. In some embodiments, the modulator is a modulator of Bcl-2, Bcl-xL, and Mcl-1.

In some embodiments, the modulator is:

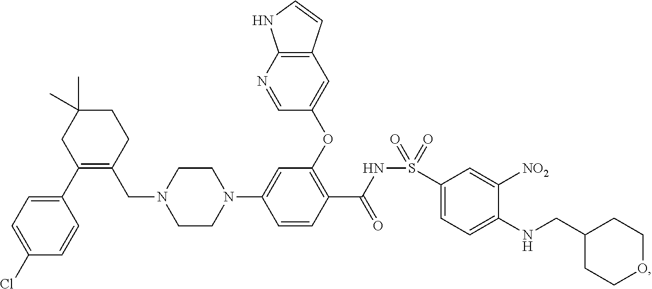 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable prodrug thereof.

In some embodiments, the modulator is:

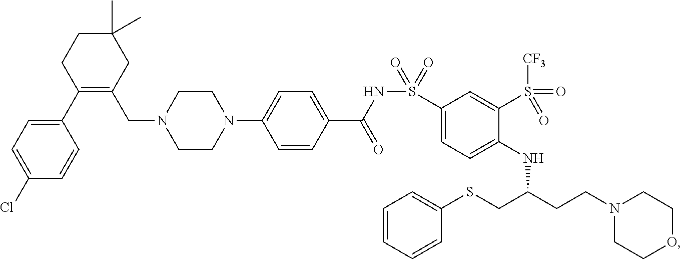 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

or a pharmaceutically acceptable prodrug thereof.

In some embodiments, the modulator is:

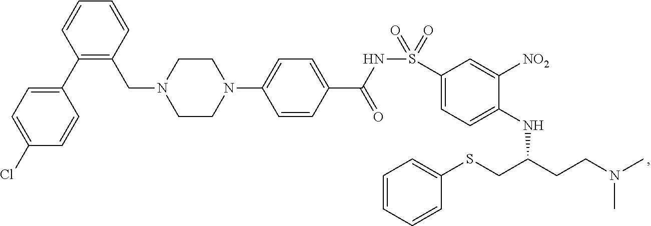 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

or a pharmaceutically acceptable prodrug thereof.

In some embodiments, the modulator is:

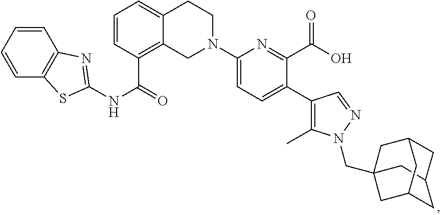 [SMILES: Cc1c(-c2ccc(N3CCc4cccc(C(=O)Nc5nc6ccccc6s5)c4C3)nc2C(=O)O)cnn1CC12CC3CC(CC(C3)C1)C2]

or a pharmaceutically acceptable prodrug thereof.

In some embodiments, the modulator is:

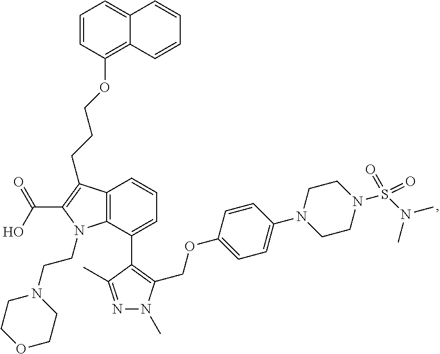 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCOCC3)c12]

or a pharmaceutically acceptable prodrug thereof.

In some embodiments, the modulator is:

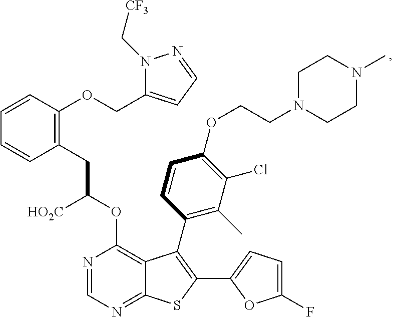

or a pharmaceutically acceptable prodrug thereof.

In some embodiments, the modulator is:

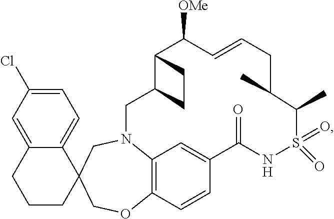 [SMILES: CO[C@H]1/C=C/C[C@H](C)[C@@H](C)S(=O)(=O)NC(=O)c2ccc3c(c2)N(C[C@@H]2CC[C@H]21)CC1(CCCc2cc(Cl)ccc21)CO3]

or a pharmaceutically acceptable prodrug thereof.

In some embodiments, the modulator is:

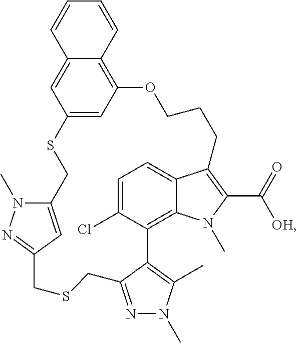 [SMILES: Cc1c2c(nn1C)CSCc1cc(n(C)n1)CSc1cc(c3ccccc3c1)OCCCc1c(C(=O)O)n(C)c3c-2c(Cl)ccc13]

or a pharmaceutically acceptable prodrug thereof.

In some embodiments, the modulator is:

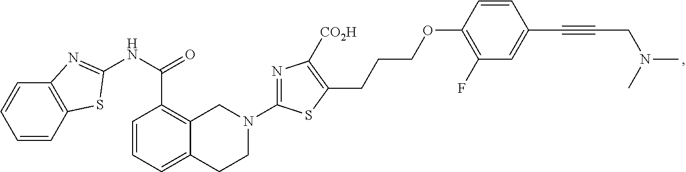 [SMILES: CN(C)CC#Cc1ccc(OCCCc2sc(N3CCc4cccc(C(=O)Nc5nc6ccccc6s5)c4C3)nc2C(=O)O)c(F)c1]

or a pharmaceutically acceptable prodrug thereof.

In some embodiments, the composition is suitable for injection. In some embodiments, the composition is suitable for intravenous administration. In some embodiments, the composition is administered intraperitoneally, intraarterially, intrapulmonarily, orally, by inhalation, intravesicularly, intramuscularly, intratracheally, subcutaneously, intraocularly, intrathecally, intratumorally, or transdermally.

Provided herein in another aspect is a method of treating a disease in a subject in need thereof comprising administering the composition comprising nanoparticles, wherein the nanoparticles comprise a modulator of a Bcl-2 family protein; and a pharmaceutically acceptable carrier; wherein the pharmaceutically acceptable carrier comprises albumin.

In some embodiments, the disease is cancer. In some embodiments, the cancer is bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer. In some embodiments, the cancer is chronic lymphocytic leukemia. In some embodiments, the cancer is small lymphocytic lymphoma, acute lymphocytic leukemia, or acute myeloid leukemia.

In some embodiments, the disease is solid tumor. In some embodiments, the solid tumor is a cancer of the brain, breast, cervix, colon, kidney, larynx, lung, ovary, pancreas, prostate, rectum, skin, spine, stomach, or uterus. In some embodiments, the solid tumor is a soft tissue sarcoma.

Provided in another aspect is a method of delivering a modulator of a Bcl-2 family protein to a subject in need thereof comprising administering any one of the compositions described herein.

Provided in another aspect is a process of preparing any one of the compositions described herein comprising
a) dissolving a modulator of a Bcl-2 family protein in a volatile solvent to form a solution comprising a dissolved modulator of a Bcl-2 family protein;
b) adding the solution comprising the dissolved modulator of a Bcl-2 family protein to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form any one of the compositions described herein.

In some embodiments, the volatile solvent is a chlorinated solvent, alcohol, ketone, ester, ether, acetonitrile, or any combination thereof. In some embodiments, the volatile solvent is chloroform, ethanol, methanol, or butanol. In some embodiments, the homogenization is high pressure homogenization. In some embodiments, the emulsion is cycled through high pressure homogenization for an appropriate amount of cycles. In some embodiments, the appropriate amount of cycles is from about 2 to about 10 cycles. In some embodiments, the evaporation is accomplished with a rotary evaporator. In some embodiments, the evaporation is under reduced pressure.

### DETAILED DESCRIPTION OF THE DISCLOSURE

This application recognizes the use of nanoparticles as a drug delivery platform is an attractive approach as nanoparticles provide the following advantages: more specific drug targeting and delivery, reduction in toxicity while maintaining therapeutic effects, greater safety and biocompatibility, and faster development of new safe medicines. The use of a pharmaceutically acceptable carrier, such as a protein, is also advantageous as proteins, such as albumin, are nontoxic, non-immunogenic, biocompatible, and biodegradable.

This application also recognizes that the overexpression of Bcl-2 family has been implicated in different malignancies. Accordingly, the development of new therapies that target the Bcl-2 family of proteins for treating cancer is an evolving area of research.

Provided herein are compositions comprising nanoparticles that allow for the drug delivery of the compounds described herein, which are modulators of the B cell lymphoma (Bcl-2) family proteins, such as Bcl-2, Bcl-xL, Bcl-w, and Mcl-1. These nanoparticle compositions further comprise pharmaceutically acceptable carriers that interact with the compounds described herein to provide the compositions in a form that is suitable for administration to a subject in need thereof. In some embodiments, this application recognizes that the modulators of the Bcl-2 family proteins, such as any one of the compounds described herein, with specific pharmaceutically acceptable carriers, such as the albumin-based pharmaceutically acceptable carriers described herein, provide nanoparticle formulations that are stable.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” or “alkylene” refers to a straight or branched hydrocarbon chain radical consisting solely of carbon and hydrogen atoms, containing no unsaturation, having from one to eighteen carbon atoms (e.g., C₁-C₁₈ alkyl). In certain embodiments, an alkyl comprises three to eighteen carbon atoms (e.g., C₃-C₁₈ alkyl). In certain embodiments, an alkyl comprises one to fifteen carbon atoms (e.g., C₁-C₁₅ alkyl). In certain embodiments, an alkyl comprises one to twelve carbon atoms (e.g., C₁-C₁₂ alkyl). In certain embodiments, an alkyl comprises one to eight carbon atoms (e.g., C₁-C₈ alkyl). In other embodiments, an alkyl comprises one to six carbon atoms (e.g., C₁-C₆ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜ^{f} (where t is 1 or 2) and —S(O)N(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to eighteen carbon atoms. In certain embodiments, an alkenyl comprises three to eighteen carbon atoms. In certain embodiments, an alkenyl comprises three to twelve carbon atoms. In certain embodiments, an alkenyl comprises six to twelve carbon atoms. In certain embodiments, an alkenyl comprises six to ten carbon atoms. In certain embodiments, an alkenyl comprises eight to ten carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)N(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Alkynyl” refers to a straight or branched hydrocarbon chain radical group consisting solely of carbon and hydrogen atoms, containing at least one carbon-carbon triple bond, having from two to eighteen carbon atoms. In certain embodiments, an alkynyl comprises three to eighteen carbon atoms. In certain embodiments, an alkynyl comprises three to twelve carbon atoms. In certain embodiments, an alkynyl comprises six to twelve carbon atoms. In certain embodiments, an alkynyl comprises six to ten carbon atoms. In certain embodiments, an alkynyl comprises eight to ten carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)N(R^{a})₂ (where t is 1 or 2) where each R^{a} is independently hydrogen, alkyl, haloalkyl, cycloalkyl, aryl, aralkyl, heterocycloalkyl, heteroaryl, or heteroarylalkyl, and each R^{f} is independently alkyl, haloalkyl, cycloalkyl, aryl, aralkyl, heterocycloalkyl, heteroaryl, or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)N(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“Aryloxy” refers to a radical bonded through an oxygen atom of the formula —O-aryl, where aryl is as defined above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkyloxy” refers to a radical bonded through an oxygen atom of the formula —O-aralkyl, where aralkyl is as defined above.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. The cycloalkyl is attached to the rest of the molecule by a single bond. Cycloalkyls are saturated, (i.e., containing single C—C bonds only) or partially unsaturated (i.e., containing one or more double bonds or triple bonds.) Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). A partially unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless stated otherwise specifically in the specification, the term “cycloalkyl” is meant to include cycloalkyl radicals that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“Halo” or “halogen” refers to bromo, chloro, fluoro or iodo substituents.

“Haloalkyl” refers to an alkyl radical, as defined above, that is substituted by one or more halo radicals, as defined above.

“Haloalkoxy” refers to an alkoxy radical, as defined above, that is substituted by one or more halo radicals, as defined above.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. The alkyl part of the fluoroalkyl radical are optionally substituted as defined above for an alkyl group.

“Heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocycloalkyl radical is a monocyclic, bicyclic, tricyclic, or tetracyclic ring system, which include fused, spiro, or bridged ring systems. The heteroatoms in the heterocycloalkyl radical are optionally oxidized. One or more nitrogen atoms, if present, are optionally quaternized. The heterocycloalkyl radical is partially or fully saturated. In some embodiments, the heterocycloalkyl is attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)OR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“Heteroaryl” refers to a radical derived from a 3- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above which are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, haloalkyl, cycloalkyl, cycloalkylalkyl, aryl, aralkyl, heterocycloalkyl, heteroaryl, or heteroarylalkyl, each R^{b} is independently a direct bond or a straight or branched alkylene or alkenylene chain, and R^{c} is a straight or branched alkylene or alkenylene chain.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O— heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, the compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

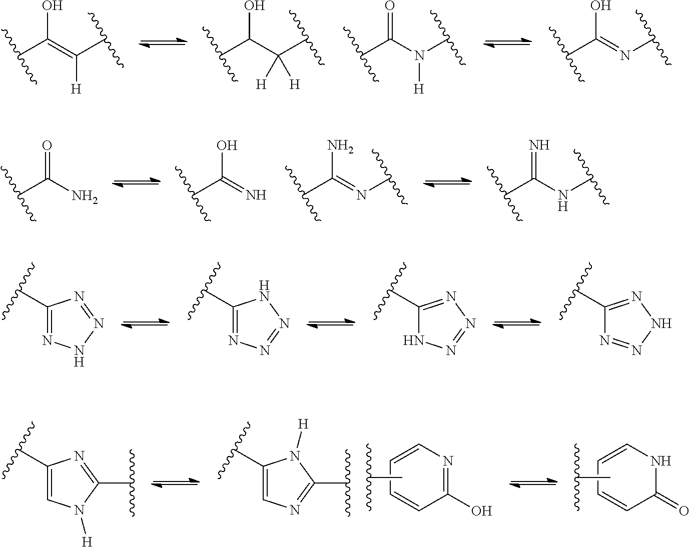

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist.

“Optional” or “optionally” means that a subsequently described event or circumstance may or may not occur and that the description includes instances when the event or circumstance occurs and instances in which it does not. For example, “optionally substituted aryl” means that the aryl radical are or are not substituted and that the description includes both substituted aryl radicals and aryl radicals having no substitution.

“Prodrug” is meant to indicate a compound that is converted under physiological conditions or by solvolysis to a biologically active compound described herein. Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility, or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. In some embodiments, prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino, or free mercapto group, respectively. Examples of prodrugs include any suitable derivatives of alcohol or amine functional groups in the active compounds and the like that are known to a skilled practitioner. Examples of any suitable derivatives include but are not limited to acetate, formate, and benzoate derivatives of alcohol or amine functional groups.

As used herein, “treatment” or “treating” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Bcl-2

The B-cell lymphoma-2 (Bcl-2) family of proteins plays a role in the regulation of apoptosis. The members of the Bcl-2 protein family are characterized into two groups according to their pro- and anti-apoptotic effects. The anti-apoptotic proteins are Bcl-2, Bcl-xL, Mcl-1, Bcl-w, Bcl-b (also known as Bcl-2-like 10), and A1 (also known as Bfl-1). The pro-apoptotic proteins are Bax, Bak, Bok, Bad, Bim, Puma, Bid, Bik, Noxa, Hrk, and Bmf.

The Bcl-2 family members have up to four conserved Bcl-2 homology (BH) domains, which are designated BH1, BH2, BH3, and BH4 and correspond to α-helical segments. Many of the anti-apoptotic protein display sequence conservation in all four domains. Pro-apoptotic proteins are subdivided into two classes according to their BH domains: multi-domain members, such as Bax, Bak and Bok, which contain and share homology in the BH1, BH2, BH3, and BH4 domains; and BH3-only proteins, including Bad, Bim, Puma, Bid, Bik, Noxa, Hrk, and Bmf, which show homology only in the BH3 domain. Many Bcl-2 family members also contain a carboxy-terminal hydrophobic domain, which has been implicated in the targeting of mitochondrial outer membrane for Bcl-2.

The compositions described herein comprise compounds that are modulators of the B-cell lymphoma-2 (Bcl-2) family proteins. The modulators of the Bcl-2 family proteins include the anti-apoptotic proteins, such as Bcl-2, Bcl-xL, Mcl-1, Bcl-w, Bcl-b, and A1, and pro-apoptotic proteins, such as Bax, Bak, Bok, Bad, Bim, Puma, Bid, Bik, Noxa, Hrk, and Bmf.

In some embodiments, the modulators include those that modulate Bcl-2, Bcl-xL, Bcl-w, Bcl-b, A1, and/or Mcl-1. In some embodiments, the modulators include those that modulate of Bcl-2, Bcl-xL, Bcl-w, and/or Mcl-1. In some embodiments, the modulators include those that modulate Bcl-2, Bcl-xL, and/or Mcl-1.

In some embodiments, the modulator is a modulator of Bcl-2. In some embodiments, the modulator is a modulator of Bcl-xL. In some embodiments, the modulator is a modulator of Bcl-w. In some embodiments, the modulator is a modulator of Bcl-b. In some embodiments, the modulator is a modulator of A1. In some embodiments, the modulator is an modulator of Mcl-1. In some embodiments, the modulator is a modulator of Bcl-2 and Bcl-xL. In some embodiments, the modulator is a modulator of Bcl-2, Bcl-xL, and Bcl-w. In some embodiments, the modulator is a modulator of Bcl-2, Bcl-xL, and Mcl-1. In some embodiments, the modulator is a modulator of Bcl-2, Bcl-xL, Bcl-w, and Mcl-1. In some embodiments, the modulator is a modulator of any combination of Bcl-2, Bcl-xL, Bcl-w, Bcl-b, A1, and Mcl-1.

In some embodiments, the compound is:

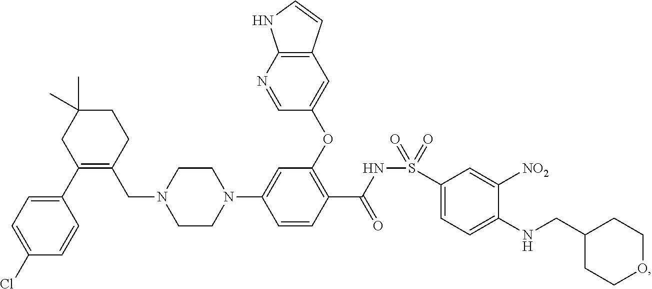 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable prodrug thereof.

In some embodiments, the compound is:

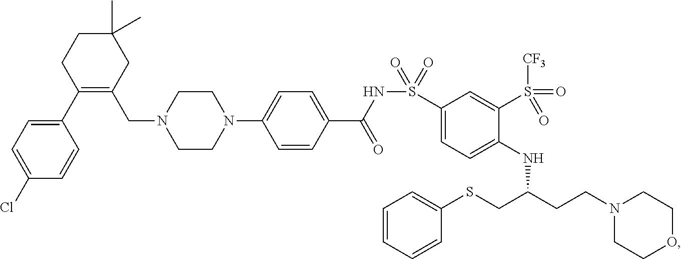 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

or a pharmaceutically acceptable prodrug thereof.

In some embodiments, the compound is:

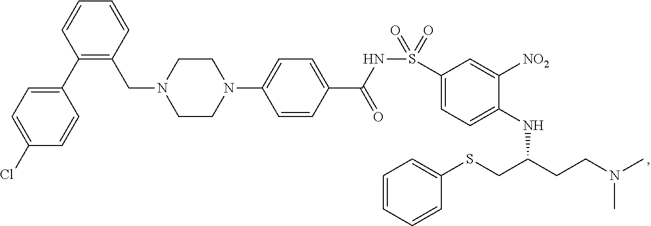 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

or a pharmaceutically acceptable prodrug thereof.

In some embodiments, the compound is:

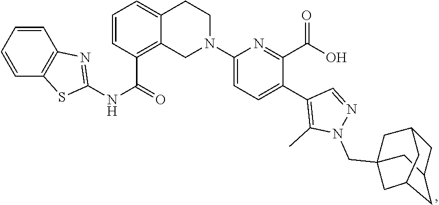 [SMILES: Cc1c(-c2ccc(N3CCc4cccc(C(=O)Nc5nc6ccccc6s5)c4C3)nc2C(=O)O)cnn1CC12CC3CC(CC(C3)C1)C2]

or a pharmaceutically acceptable prodrug thereof.

In some embodiments, the compound is:

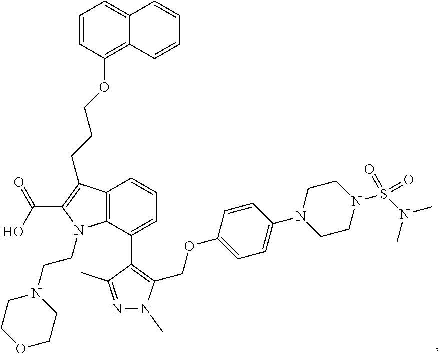 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCOCC3)c12]

or a pharmaceutically acceptable prodrug thereof.

In some embodiments, wherein the compound is:

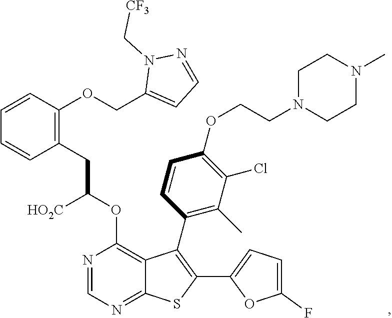

or a pharmaceutically acceptable prodrug thereof.

In some embodiments, wherein the compound is:

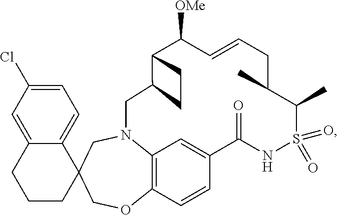 [SMILES: CO[C@H]1/C=C/C[C@H](C)[C@@H](C)S(=O)(=O)NC(=O)c2ccc3c(c2)N(C[C@@H]2CC[C@H]21)CC1(CCCc2cc(Cl)ccc21)CO3]

or a pharmaceutically acceptable prodrug thereof.

In some embodiments, wherein the compound is:

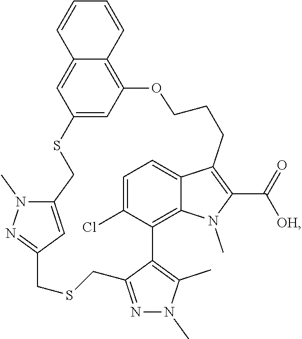 [SMILES: Cc1c2c(nn1C)CSCc1cc(n(C)n1)CSc1cc(c3ccccc3c1)OCCCc1c(C(=O)O)n(C)c3c-2c(Cl)ccc13]

or a pharmaceutically acceptable prodrug thereof.

In some embodiments, the modulator is:

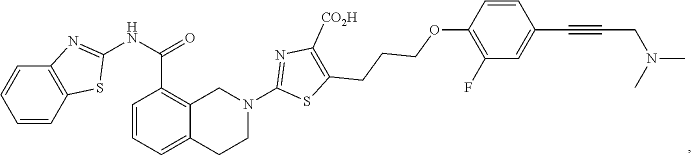 [SMILES: CN(C)CC#Cc1ccc(OCCCc2sc(N3CCc4cccc(C(=O)Nc5nc6ccccc6s5)c4C3)nc2C(=O)O)c(F)c1]

or a pharmaceutically acceptable prodrug thereof.

In some embodiments, the compound is any one of the compounds shown in the following table:

| Compound | |
|---|---|
| No. | Structure |
| 1 | 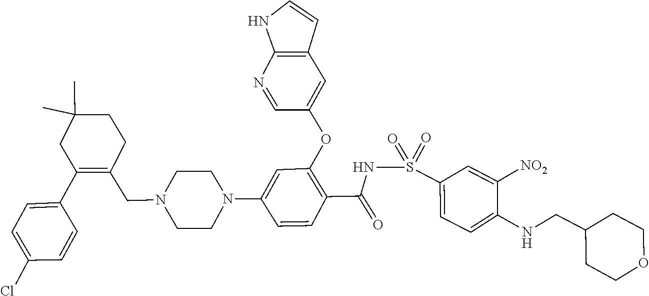 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1] |
| 2 | 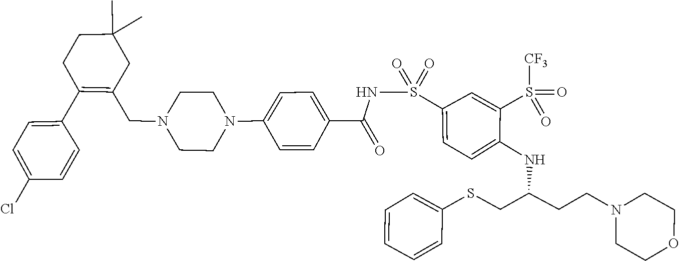 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1] |
| 3 | 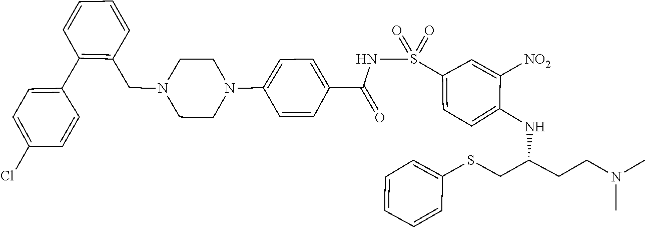 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]] |
| 4 | 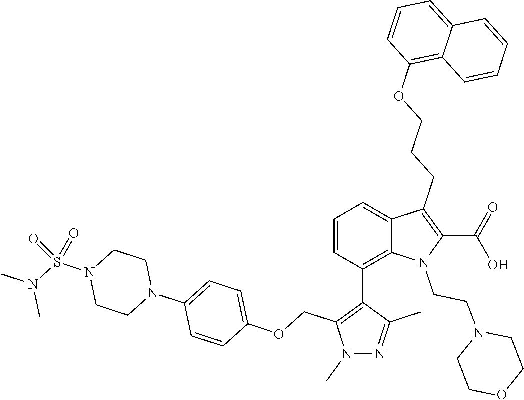 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCOCC3)c12] |
| 5 | 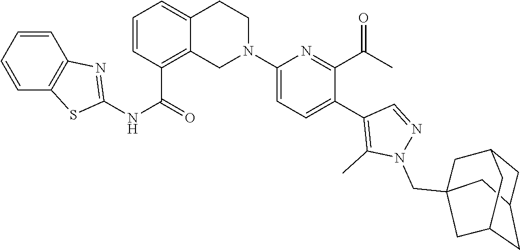 [SMILES: CC(=O)c1nc(N2CCc3cccc(C(=O)Nc4nc5ccccc5s4)c3C2)ccc1-c1cnn(CC23CC4CC(CC(C4)C2)C3)c1C] |
| 6 | 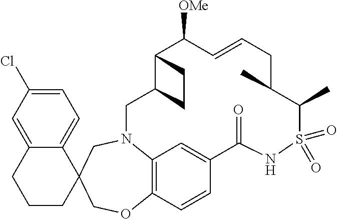 [SMILES: CO[C@H]1/C=C/C[C@H](C)[C@@H](C)S(=O)(=O)NC(=O)c2ccc3c(c2)N(C[C@@H]2CC[C@H]21)CC1(CCCc2cc(Cl)ccc21)CO3] |
| 7 | 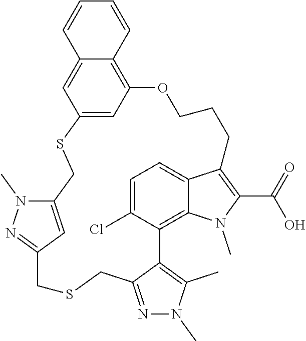 [SMILES: Cc1c2c(nn1C)CSCc1cc(n(C)n1)CSc1cc(c3ccccc3c1)OCCCc1c(C(=O)O)n(C)c3c-2c(Cl)ccc13] |
| 8 | 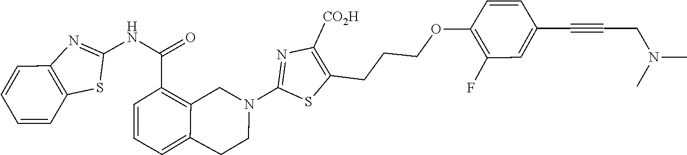 [SMILES: CN(C)CC#Cc1ccc(OCCCc2sc(N3CCc4cccc(C(=O)Nc5nc6ccccc6s5)c4C3)nc2C(=O)O)c(F)c1] |

Examples of compounds that modulate the Bcl-2 family proteins include compounds disclosed in the following publications: Azmi, et. al., Emerging Bcl-2 inhibitors for the treatment of cancer, Expert Opin Emerg. Drug. 2011, 16(1), p 59-70; and Bajwa, et. al., Inhibitors of the anti-apoptotic Bcl-2 proteins: a patent review, Expert Opin Ther Pat. 2012, 22(1), 37-55; which are incorporated by reference for disclosure of such compounds.

In some embodiments, a compound that modulates Bcl-2 family proteins is a compound that non-selectively or selectively inhibits the activity of one or more Bcl-2 family protein members In some embodiments, the one or more Bcl-2 family protein member is Bcl-2, Bcl-xL, Mcl-1, Bcl-w, Bcl-b, A1 or any combination thereof. In some embodiments, the one or more Bcl-2 family protein member is Bcl-2. In some embodiments, the one or more Bcl-2 family protein member is Bcl-xL. In some embodiments, the one or more Bcl-2 family protein member is Mcl-1. In some embodiments, the one or more Bcl-2 family protein member is Bcl-w. In some embodiments, the one or more Bcl-2 family protein member is Bcl-b. In some embodiments, the one or more Bcl-2 family protein member is A1. In some embodiments, the one or more Bcl-2 family protein member is Bcl-2 and Bcl-xL. In some embodiments, the one or more Bcl-2 family protein member is Bcl-2, Bcl-xL, and Bcl-w. In some embodiments, the one or more Bcl-2 family protein member is Bcl-2, Bcl-xL, and Mcl-1. In some embodiments, the one or more Bel-2 family protein member is Bcl-2, Bcl-xL, Bcl-w, and Mcl-1.

In some embodiments, the compound has an inhibitory potency (IC₅₀) of less than about 100 nanomolar. In some embodiments, the compound has an inhibitory potency (IC₅₀) of less than about 50 nanomolar. In some embodiments, the compound has an inhibitory potency (IC₅₀) of less than about 10 nanomolar. In some embodiments, the compound has an inhibitory potency (IC₅₀) of less than about 1 nanomolar.

### Preparation of Compounds

The compounds used in the reactions described herein are made according to organic synthesis techniques, starting from commercially available chemicals and/or from compounds described in the chemical literature. “Commercially available chemicals” are obtained from standard commercial sources include, but are not limited to, Acros Organics (Geel, Belgium), Aldrich Chemical (Milwaukee, Wis., including Sigma Chemical and Fluka), Apin Chemicals Ltd. (Milton Park, UK), Ark Pharm, Inc. (Libertyville, Ill.), Avocado Research (Lancashire, U.K.), BDH (Toronto, Canada), Bionet (Cornwall, U.K.), Chemietek (Indianapolis, Ind.), Chemservice Inc. (West Chester, Pa.), Combi-blocks (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), MedChemExpress (Monmouth Junction, N.J.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Spectrum Chemicals (Gardena, Calif.), Sundia Meditech, (Shanghai, China), TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C.). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization. In some embodiments, the compounds described herein exist as rotational isomers. The compounds presented herein include all rotational isomers as well as the corresponding mixtures thereof.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or MeOH. In addition, the compounds provided herein exist in unsolvated as well as solvated forms. In general, the solvated forms are considered equivalent to the unsolvated forms for the purposes of the compounds and methods provided herein.

### Prodrugs

In some embodiments, compounds described herein are prepared as prodrugs. A “prodrug” refers to an agent that is converted into the parent drug in vivo. Prodrugs are often useful because, in some situations, they are easier to administer than the parent drug. In some embodiments, the prodrug is a substrate for a transporter. In some embodiments, the prodrug also has improved solubility in pharmaceutical compositions over the parent drug. In some embodiments, the design of a prodrug increases the effective water solubility. In some embodiments, the design of a prodrug decreases the effective water solubility. An example, without limitation, of a prodrug is a compound described herein, which is administered as an ester (the “prodrug”) but then is metabolically hydrolyzed to provide the active entity. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound.

### Metabolites

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutically Acceptable Carrier

In some embodiments, the composition described herein also comprise a pharmaceutically acceptable carrier. In some embodiments, the pharmaceutically acceptable carrier is a protein. The term “protein’ as used herein refers to polypeptides or polymers comprising of amino acids of any length (including full length or fragments). These polypeptides or polymers are linear or branched, comprise modified amino acids, and/or are interrupted by non-amino acids. The term also encompasses an amino acid polymer that has been modified by natural means or by chemical modification. Examples of chemical modifications include, but are not limited to, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The proteins described herein may be naturally occurring, i.e., obtained or derived from a natural source (such as blood), or synthesized (such as chemically synthesized or synthesized by recombinant DNA techniques). In some embodiments, the protein is naturally occurring. In some embodiments, the protein is obtained or derived from a natural source. In some embodiments, the protein is synthetically prepared.

Examples of suitable pharmaceutically acceptable carriers include proteins normally found in blood or plasma, such as albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the pharmaceutically acceptable carrier is a non-blood protein. Examples of non-blood protein include but are not limited to casein, C.-lactalbumin, and B-lactoglobulin.

In some embodiments, the pharmaceutically acceptable carrier is albumin. In some embodiments, the albumin is human serum albumin (HSA). Human serum albumin is the most abundant protein in human blood and is a highly soluble globular protein that consists of 585 amino acids and has a molecular weight of 66.5 kDa. Other albumins suitable for use include, but are not limited to, bovine serum albumin.

In some non-limiting embodiments, the composition described herein further comprises one or more albumin stabilizers. In some embodiments, the albumin stabilizer is N-acetyl tryptophan, octanoate salts, or a combination thereof.

In some embodiments, the molar ratio of the compound to pharmaceutically acceptable carrier is from about 1:1 to about 40:1. In some embodiments, the molar ratio of the compound to pharmaceutically acceptable carrier is from about 1:1 to about 20:1. In some embodiments, the molar ratio of the compound to pharmaceutically acceptable carrier is from about 2:1 to about 12:1.

In some embodiments, the molar ratio of the compound to pharmaceutically acceptable carrier is about 40:1. In some embodiments, the molar ratio of the compound to pharmaceutically acceptable carrier is about 35:1. In some embodiments, the molar ratio of the compound to pharmaceutically acceptable carrier is about 30:1. In some embodiments, the molar ratio of the compound to pharmaceutically acceptable carrier is about 25:1. In some embodiments, the molar ratio of the compound to pharmaceutically acceptable carrier is about 20:1. In some embodiments, the molar ratio of the compound to pharmaceutically acceptable carrier is about 19:1. In some embodiments, the molar ratio of the compound to pharmaceutically acceptable carrier is about 18:1. In some embodiments, the molar ratio of the compound to pharmaceutically acceptable carrier is about 17:1. In some embodiments, the molar ratio of the compound to pharmaceutically acceptable carrier is about 16:1. In some embodiments, the molar ratio of the compound to pharmaceutically acceptable carrier is about 15:1. In some embodiments, the molar ratio of the compound to pharmaceutically acceptable carrier is about 14:1. In some embodiments, the molar ratio of the compound to pharmaceutically acceptable carrier is about 13:1. In some embodiments, the molar ratio of the compound to pharmaceutically acceptable carrier is about 12:1. In some embodiments, the molar ratio of the compound to pharmaceutically acceptable carrier is about 11:1. In some embodiments, the molar ratio of the compound to pharmaceutically acceptable carrier is about 10:1. In some embodiments, the molar ratio of the compound to pharmaceutically acceptable carrier is about 9:1. In some embodiments, the molar ratio of the compound to pharmaceutically acceptable carrier is about 8:1. In some embodiments, the molar ratio of the compound to pharmaceutically acceptable carrier is about 7:1. In some embodiments, the molar ratio of the compound to pharmaceutically acceptable carrier is about 6:1. In some embodiments, the molar ratio of the compound to pharmaceutically acceptable carrier is about 5:1. In some embodiments, the molar ratio of the compound to pharmaceutically acceptable carrier is about 4:1. In some embodiments, the molar ratio of the compound to pharmaceutically acceptable carrier is about 3:1. In some embodiments, the molar ratio of the compound to pharmaceutically acceptable carrier is about 2:1.

### Nanoparticles

Described herein in one aspect is a composition comprising nanoparticles comprising any one of the compounds described herein, which are modulators of the Bcl-2 family proteins; and a pharmaceutically acceptable carrier.

In some embodiments, the nanoparticles have an average diameter of about 1000 nm or less for a predetermined amount of time after nanoparticle formation. In some embodiments, the nanoparticles have an average diameter of about 950 nm or less for a predetermined amount of time after nanoparticle formation. In some embodiments, the nanoparticles have an average diameter of about 900 nm or less for a predetermined amount of time after nanoparticle formation. In some embodiments, the nanoparticles have an average diameter of about 850 nm or less for a predetermined amount of time after nanoparticle formation. In some embodiments, the nanoparticles have an average diameter of about 800 nm or less for a predetermined amount of time after nanoparticle formation. In some embodiments, the nanoparticles have an average diameter of about 750 nm or less for a predetermined amount of time after nanoparticle formation. In some embodiments, the nanoparticles have an average diameter of about 700 nm or less for a predetermined amount of time after nanoparticle formation. In some embodiments, the nanoparticles have an average diameter of about 650 nm or less for a predetermined amount of time after nanoparticle formation. In some embodiments, the nanoparticles have an average diameter of about 600 nm or less for a predetermined amount of time after nanoparticle formation. In some embodiments, the nanoparticles have an average diameter of about 550 nm or less for a predetermined amount of time after nanoparticle formation. In some embodiments, the nanoparticles have an average diameter of about 500 nm or less for a predetermined amount of time after nanoparticle formation. In some embodiments, the nanoparticles have an average diameter of about 450 nm or less for a predetermined amount of time after nanoparticle formation. In some embodiments, the nanoparticles have an average diameter of about 400 nm or less for a predetermined amount of time after nanoparticle formation. In some embodiments, the nanoparticles have an average diameter of about 350 nm or less for a predetermined amount of time after nanoparticle formation. In some embodiments, the nanoparticles have an average diameter of about 300 nm or less for a predetermined amount of time after nanoparticle formation. In some embodiments, the nanoparticles have an average diameter of about 250 nm or less for a predetermined amount of time after nanoparticle formation. In some embodiments, the nanoparticles have an average diameter of about 240 nm or less for a predetermined amount of time after nanoparticle formation. In some embodiments, the nanoparticles have an average diameter of about 230 nm or less for a predetermined amount of time after nanoparticle formation. In some embodiments, the nanoparticles have an average diameter of about 220 nm or less for a predetermined amount of time after nanoparticle formation. In some embodiments, the nanoparticles have an average diameter of about 210 nm or less for a predetermined amount of time after nanoparticle formation. In some embodiments, the nanoparticles have an average diameter of about 200 nm or less for a predetermined amount of time after nanoparticle formation. In some embodiments, the nanoparticles have an average diameter of about 190 nm or less for a predetermined amount of time after nanoparticle formation. In some embodiments, the nanoparticles have an average diameter of about 180 nm or less for a predetermined amount of time after nanoparticle formation. In some embodiments, the nanoparticles have an average diameter of about 170 nm or less for a predetermined amount of time after nanoparticle formation. In some embodiments, the nanoparticles have an average diameter of about 160 nm or less for a predetermined amount of time after nanoparticle formation. In some embodiments, the nanoparticles have an average diameter of about 150 nm or less for a predetermined amount of time after nanoparticle formation. In some embodiments, the nanoparticles have an average diameter of about 140 nm or less for a predetermined amount of time after nanoparticle formation. In some embodiments, the nanoparticles have an average diameter of about 130 nm or less for a predetermined amount of time after nanoparticle formation. In some embodiments, the nanoparticles have an average diameter of about 120 nm or less for a predetermined amount of time after nanoparticle formation. In some embodiments, the nanoparticles have an average diameter of about 110 nm or less for a predetermined amount of time after nanoparticle formation. In some embodiments, the nanoparticles have an average diameter of about 100 nm or less for a predetermined amount of time after nanoparticle formation. In some embodiments, the nanoparticles have an average diameter of about 90 nm or less for a predetermined amount of time after nanoparticle formation. In some embodiments, the nanoparticles have an average diameter of about 80 nm or less for a predetermined amount of time after nanoparticle formation. In some embodiments, the nanoparticles have an average diameter of about 70 nm or less for a predetermined amount of time after nanoparticle formation. In some embodiments, the nanoparticles have an average diameter of about 60 nm or less for a predetermined amount of time after nanoparticle formation. In some embodiments, the nanoparticles have an average diameter of about 50 nm or less for a predetermined amount of time after nanoparticle formation. In some embodiments, the nanoparticles have an average diameter of about 40 nm or less for a predetermined amount of time after nanoparticle formation. In some embodiments, the nanoparticles have an average diameter of about 30 nm or less for a predetermined amount of time after nanoparticle formation. In some embodiments, the nanoparticles have an average diameter of about 20 nm or less for a predetermined amount of time after nanoparticle formation. In some embodiments, the nanoparticles have an average diameter of about 10 nm or less for a predetermined amount of time after nanoparticle formation.

In some embodiments, the nanoparticles have an average diameter of about 10 nm or greater for a predetermined amount of time after nanoparticle formation. In some embodiments, the nanoparticles have an average diameter of about 20 nm or greater for a predetermined amount of time after nanoparticle formation. In some embodiments, the nanoparticles have an average diameter of about 30 nm or greater for a predetermined amount of time after nanoparticle formation. In some embodiments, the nanoparticles have an average diameter of about 40 nm or greater for a predetermined amount of time after nanoparticle formation. In some embodiments, the nanoparticles have an average diameter of about 50 nm or greater for a predetermined amount of time after nanoparticle formation. In some embodiments, the nanoparticles have an average diameter of about 60 nm or greater for a predetermined amount of time after nanoparticle formation. In some embodiments, the nanoparticles have an average diameter of about 70 nm or greater for a predetermined amount of time after nanoparticle formation. In some embodiments, the nanoparticles have an average diameter of about 80 nm or greater for a predetermined amount of time after nanoparticle formation. In some embodiments, the nanoparticles have an average diameter of about 90 nm or greater for a predetermined amount of time after nanoparticle formation. In some embodiments, the nanoparticles have an average diameter of about 100 nm or greater for a predetermined amount of time after nanoparticle formation. In some embodiments, the nanoparticles have an average diameter of about 110 nm or greater for a predetermined amount of time after nanoparticle formation. In some embodiments, the nanoparticles have an average diameter of about 120 nm or greater for a predetermined amount of time after nanoparticle formation. In some embodiments, the nanoparticles have an average diameter of about 130 nm or greater for a predetermined amount of time after nanoparticle formation. In some embodiments, the nanoparticles have an average diameter of about 140 nm or greater for a predetermined amount of time after nanoparticle formation. In some embodiments, the nanoparticles have an average diameter of about 150 nm or greater for a predetermined amount of time after nanoparticle formation. In some embodiments, the nanoparticles have an average diameter of about 160 nm or greater for a predetermined amount of time after nanoparticle formation. In some embodiments, the nanoparticles have an average diameter of about 170 nm or greater for a predetermined amount of time after nanoparticle formation. In some embodiments, the nanoparticles have an average diameter of about 180 nm or greater for a predetermined amount of time after nanoparticle formation. In some embodiments, the nanoparticles have an average diameter of about 190 nm or greater for a predetermined amount of time after nanoparticle formation. In some embodiments, the nanoparticles have an average diameter of about 200 nm or greater for a predetermined amount of time after nanoparticle formation. In some embodiments, the nanoparticles have an average diameter of about 210 nm or greater for a predetermined amount of time after nanoparticle formation. In some embodiments, the nanoparticles have an average diameter of about 220 nm or greater for a predetermined amount of time after nanoparticle formation. In some embodiments, the nanoparticles have an average diameter of about 230 nm or greater for a predetermined amount of time after nanoparticle formation. In some embodiments, the nanoparticles have an average diameter of about 240 nm or greater for a predetermined amount of time after nanoparticle formation. In some embodiments, the nanoparticles have an average diameter of about 250 nm or greater for a predetermined amount of time after nanoparticle formation. In some embodiments, the nanoparticles have an average diameter of about 300 nm or greater for a predetermined amount of time after nanoparticle formation. In some embodiments, the nanoparticles have an average diameter of about 350 nm or greater for a predetermined amount of time after nanoparticle formation. In some embodiments, the nanoparticles have an average diameter of about 400 nm or greater for a predetermined amount of time after nanoparticle formation. In some embodiments, the nanoparticles have an average diameter of about 450 nm or greater for a predetermined amount of time after nanoparticle formation. In some embodiments, the nanoparticles have an average diameter of about 500 nm or greater for a predetermined amount of time after nanoparticle formation. In some embodiments, the nanoparticles have an average diameter of about 550 nm or greater for a predetermined amount of time after nanoparticle formation. In some embodiments, the nanoparticles have an average diameter of about 600 nm or greater for a predetermined amount of time after nanoparticle formation. In some embodiments, the nanoparticles have an average diameter of about 650 nm or greater for a predetermined amount of time after nanoparticle formation. In some embodiments, the nanoparticles have an average diameter of about 700 nm or greater for a predetermined amount of time after nanoparticle formation. In some embodiments, the nanoparticles have an average diameter of about 750 nm or greater for a predetermined amount of time after nanoparticle formation. In some embodiments, the nanoparticles have an average diameter of about 800 nm or greater for a predetermined amount of time after nanoparticle formation. In some embodiments, the nanoparticles have an average diameter of about 850 nm or greater for a predetermined amount of time after nanoparticle formation. In some embodiments, the nanoparticles have an average diameter of about 900 nm or greater for a predetermined amount of time after nanoparticle formation. In some embodiments, the nanoparticles have an average diameter of about 950 nm or greater for a predetermined amount of time after nanoparticle formation

In some embodiments, the nanoparticles have an average diameter of from about 10 nm to about 1000 nm for a predetermined amount of time after nanoparticle formation. In some embodiments, the nanoparticles have an average diameter of from about 10 nm to about 950 nm for a predetermined amount of time after nanoparticle formation. In some embodiments, the nanoparticles have an average diameter of from about 10 nm to about 900 nm for a predetermined amount of time after nanoparticle formation. In some embodiments, the nanoparticles have an average diameter of from about 10 nm to about 850 nm for a predetermined amount of time after nanoparticle formation. In some embodiments, the nanoparticles have an average diameter of from about 10 nm to about 800 nm for a predetermined amount of time after nanoparticle formation. In some embodiments, the nanoparticles have an average diameter of from about 10 nm to about 750 nm for a predetermined amount of time after nanoparticle formation. In some embodiments, the nanoparticles have an average diameter of from about 10 nm to about 700 nm for a predetermined amount of time after nanoparticle formation. In some embodiments, the nanoparticles have an average diameter of from about 10 nm to about 650 nm for a predetermined amount of time after nanoparticle formation. In some embodiments, the nanoparticles have an average diameter of from about 10 nm to about 600 nm for a predetermined amount of time after nanoparticle formation. In some embodiments, the nanoparticles have an average diameter of from about 10 nm to about 550 nm for a predetermined amount of time after nanoparticle formation for a predetermined amount of time after nanoparticle formation. In some embodiments, the nanoparticles have an average diameter of from about 10 nm to about 500 nm for a predetermined amount of time after nanoparticle formation. In some embodiments, the nanoparticles have an average diameter of from about 10 nm to about 450 nm for a predetermined amount of time after nanoparticle formation. In some embodiments, the nanoparticles have an average diameter of from about 10 nm to about 400 nm for a predetermined amount of time after nanoparticle formation. In some embodiments, the nanoparticles have an average diameter of from about 10 nm to about 350 nm for a predetermined amount of time after nanoparticle formation. In some embodiments, the nanoparticles have an average diameter of from about 10 nm to about 300 nm for a predetermined amount of time after nanoparticle formation. In some embodiments, the nanoparticles have an average diameter of from about 10 nm to about 250 nm for a predetermined amount of time after nanoparticle formation. In some embodiments, the nanoparticles have an average diameter of from about 10 nm to about 240 nm for a predetermined amount of time after nanoparticle formation. In some embodiments, the nanoparticles have an average diameter of from about 10 nm to about 230 nm for a predetermined amount of time after nanoparticle formation. In some embodiments, the nanoparticles have an average diameter of from about 10 nm to about 220 nm for a predetermined amount of time after nanoparticle formation. In some embodiments, the nanoparticles have an average diameter of from about 10 nm to about 210 nm for a predetermined amount of time after nanoparticle formation. In some embodiments, the nanoparticles have an average diameter of from about 10 nm to about 200 nm for a predetermined amount of time after nanoparticle formation. In some embodiments, the nanoparticles have an average diameter of from about 10 nm to about 190 nm for a predetermined amount of time after nanoparticle formation. In some embodiments, the nanoparticles have an average diameter of from about 10 nm to about 180 nm for a predetermined amount of time after nanoparticle formation. In some embodiments, the nanoparticles have an average diameter of from about 10 nm to about 170 nm for a predetermined amount of time after nanoparticle formation. In some embodiments, the nanoparticles have an average diameter of from about 10 nm to about 160 nm for a predetermined amount of time after nanoparticle formation. In some embodiments, the nanoparticles have an average diameter of from about 10 nm to about 150 nm for a predetermined amount of time after nanoparticle formation. In some embodiments, the nanoparticles have an average diameter of from about 10 nm to about 140 nm for a predetermined amount of time after nanoparticle formation. In some embodiments, the nanoparticles have an average diameter of from about 10 nm to about 130 nm for a predetermined amount of time after nanoparticle formation. In some embodiments, the nanoparticles have an average diameter of from about 10 nm to about 120 nm for a predetermined amount of time after nanoparticle formation. In some embodiments, the nanoparticles have an average diameter of from about 10 nm to about 110 nm for a predetermined amount of time after nanoparticle formation. In some embodiments, the nanoparticles have an average diameter of from about 10 nm to about 100 nm for a predetermined amount of time after nanoparticle formation. In some embodiments, the nanoparticles have an average diameter of from about 10 nm to about 90 nm for a predetermined amount of time after nanoparticle formation. In some embodiments, the nanoparticles have an average diameter of from about 10 nm to about 80 nm for a predetermined amount of time after nanoparticle formation. In some embodiments, the nanoparticles have an average diameter of from about 10 nm to about 70 nm for a predetermined amount of time after nanoparticle formation. In some embodiments, the nanoparticles have an average diameter of from about 10 nm to about 60 nm for a predetermined amount of time after nanoparticle formation. In some embodiments, the nanoparticles have an average diameter of from about 10 nm to about 50 nm for a predetermined amount of time after nanoparticle formation. In some embodiments, the nanoparticles have an average diameter of from about 10 nm to about 40 nm for a predetermined amount of time after nanoparticle formation. In some embodiments, the nanoparticles have an average diameter of from about 10 nm to about 30 nm for a predetermined amount of time after nanoparticle formation. In some embodiments, the nanoparticles have an average diameter of from about 10 nm to about 20 nm for a predetermined amount of time after nanoparticle formation.

In some embodiments, the nanoparticles have an average diameter of about 10 nm for a predetermined amount of time after nanoparticle formation. In some embodiments, the nanoparticles have an average diameter of about 20 nm for a predetermined amount of time after nanoparticle formation. In some embodiments, the nanoparticles have an average diameter of about 30 nm for a predetermined amount of time after nanoparticle formation. In some embodiments, the nanoparticles have an average diameter of about 40 nm for a predetermined amount of time after nanoparticle formation. In some embodiments, the nanoparticles have an average diameter of about 50 nm for a predetermined amount of time after nanoparticle formation. In some embodiments, the nanoparticles have an average diameter of about 60 nm for a predetermined amount of time after nanoparticle formation. In some embodiments, the nanoparticles have an average diameter of about 70 nm for a predetermined amount of time after nanoparticle formation. In some embodiments, the nanoparticles have an average diameter of about 80 nm for a predetermined amount of time after nanoparticle formation. In some embodiments, the nanoparticles have an average diameter of about 90 nm for a predetermined amount of time after nanoparticle formation. In some embodiments, the nanoparticles have an average diameter of about 100 nm for a predetermined amount of time after nanoparticle formation. In some embodiments, the nanoparticles have an average diameter of about 110 nm for a predetermined amount of time after nanoparticle formation. In some embodiments, the nanoparticles have an average diameter of about 120 nm for a predetermined amount of time after nanoparticle formation. In some embodiments, the nanoparticles have an average diameter of about 130 nm for a predetermined amount of time after nanoparticle formation. In some embodiments, the nanoparticles have an average diameter of about 140 nm for a predetermined amount of time after nanoparticle formation. In some embodiments, the nanoparticles have an average diameter of about 150 nm for a predetermined amount of time after nanoparticle formation. In some embodiments, the nanoparticles have an average diameter of about 160 nm for a predetermined amount of time after nanoparticle formation. In some embodiments, the nanoparticles have an average diameter of about 170 nm for a predetermined amount of time after nanoparticle formation. In some embodiments, the nanoparticles have an average diameter of about 180 nm for a predetermined amount of time after nanoparticle formation. In some embodiments, the nanoparticles have an average diameter of about 190 nm for a predetermined amount of time after nanoparticle formation. In some embodiments, the nanoparticles have an average diameter of about 200 nm for a predetermined amount of time after nanoparticle formation. In some embodiments, the nanoparticles have an average diameter of about 210 nm for a predetermined amount of time after nanoparticle formation. In some embodiments, the nanoparticles have an average diameter of about 220 nm for a predetermined amount of time after nanoparticle formation. In some embodiments, the nanoparticles have an average diameter of about 230 nm for a predetermined amount of time after nanoparticle formation. In some embodiments, the nanoparticles have an average diameter of about 240 nm for a predetermined amount of time after nanoparticle formation. In some embodiments, the nanoparticles have an average diameter of about 250 nm for a predetermined amount of time after nanoparticle formation. In some embodiments, the nanoparticles have an average diameter of about 300 nm for a predetermined amount of time after nanoparticle formation. In some embodiments, the nanoparticles have an average diameter of about 350 nm for a predetermined amount of time after nanoparticle formation. In some embodiments, the nanoparticles have an average diameter of about 400 nm for a predetermined amount of time after nanoparticle formation. In some embodiments, the nanoparticles have an average diameter of about 450 nm for a predetermined amount of time after nanoparticle formation. In some embodiments, the nanoparticles have an average diameter of about 500 nm for a predetermined amount of time after nanoparticle formation. In some embodiments, the nanoparticles have an average diameter of about 550 nm for a predetermined amount of time after nanoparticle formation. In some embodiments, the nanoparticles have an average diameter of about 600 nm for a predetermined amount of time after nanoparticle formation. In some embodiments, the nanoparticles have an average diameter of about 650 nm for a predetermined amount of time after nanoparticle formation. In some embodiments, the nanoparticles have an average diameter of about 700 nm for a predetermined amount of time after nanoparticle formation. In some embodiments, the nanoparticles have an average diameter of about 750 nm for a predetermined amount of time after nanoparticle formation. In some embodiments, the nanoparticles have an average diameter of about 800 nm for a predetermined amount of time after nanoparticle formation. In some embodiments, the nanoparticles have an average diameter of about 850 nm for a predetermined amount of time after nanoparticle formation. In some embodiments, the nanoparticles have an average diameter of about 900 nm for a predetermined amount of time after nanoparticle formation. In some embodiments, the nanoparticles have an average diameter of about 950 nm for a predetermined amount of time after nanoparticle formation. In some embodiments, the nanoparticles have an average diameter of about 1000 nm for a predetermined amount of time after nanoparticle formation.

In some embodiments, the predetermined amount of time is at least about 15 minutes. In some embodiments, the predetermined amount of time is at least about 30 minutes. In some embodiments, the predetermined amount of time is at least about 45 minutes. In some embodiments, the predetermined amount of time is at least about 1 hour. In some embodiments, the predetermined amount of time is at least about 2 hours. In some embodiments, the predetermined amount of time is at least about 3 hours. In some embodiments, the predetermined amount of time is at least about 4 hours. In some embodiments, the predetermined amount of time is at least about 5 hours. In some embodiments, the predetermined amount of time is at least about 6 hours. In some embodiments, the predetermined amount of time is at least about 7 hours. In some embodiments, the predetermined amount of time is at least about 8 hours. In some embodiments, the predetermined amount of time is at least about 9 hours. In some embodiments, the predetermined amount of time is at least about 10 hours. In some embodiments, the predetermined amount of time is at least about 11 hours. In some embodiments, the predetermined amount of time is at least about 12 hours. In some embodiments, the predetermined amount of time is at least about 1 day. In some embodiments, the predetermined amount of time is at least about 2 days. In some embodiments, the predetermined amount of time is at least about 3 days. In some embodiments, the predetermined amount of time is at least about 4 days. In some embodiments, the predetermined amount of time is at least about 5 days. In some embodiments, the predetermined amount of time is at least about 6 days. In some embodiments, the predetermined amount of time is at least about 7 days. In some embodiments, the predetermined amount of time is at least about 14 days. In some embodiments, the predetermined amount of time is at least about 21 days. In some embodiments, the predetermined amount of time is at least about 30 days.

In some embodiments, the predetermined amount of time is from about 15 minutes to about 30 days. In some embodiments, the predetermined amount of time is about 30 minutes to about 30 days. In some embodiments, the predetermined amount of time is from about 45 minutes to about 30 days. In some embodiments, the predetermined amount of time is from about 1 hour to about 30 days. In some embodiments, the predetermined amount of time is from about 2 hours to about 30 days. In some embodiments, the predetermined amount of time is from about 3 hours to about 30 days. In some embodiments, the predetermined amount of time is from about 4 hours to about 30 days. In some embodiments, the predetermined amount of time is from about 5 hours to about 30 days. In some embodiments, the predetermined amount of time is from about 6 hours to about 30 days. In some embodiments, the predetermined amount of time is from about 7 hours to about 30 days. In some embodiments, the predetermined amount of time is from about 8 hours to about 30 days. In some embodiments, the predetermined amount of time is from about 9 hours to about 30 days. In some embodiments, the predetermined amount of time is from about 10 hours to about 30 days. In some embodiments, the predetermined amount of time is from about 11 hours to about 30 days. In some embodiments, the predetermined amount of time is from about 12 hours to about 30 days. In some embodiments, the predetermined amount of time is from about 1 day to about 30 days. In some embodiments, the predetermined amount of time is from about 2 days to about 30 days. In some embodiments, the predetermined amount of time is from about 3 days to about 30 days. In some embodiments, the predetermined amount of time is from about 4 days to about 30 days. In some embodiments, the predetermined amount of time is from about 5 days to about 30 days. In some embodiments, the predetermined amount of time is from about 6 days to about 30 days. In some embodiments, the predetermined amount of time is from about 7 days to about 30 days. In some embodiments, the predetermined amount of time is from about 14 days to about 30 days. In some embodiments, the predetermined amount of time is from about 21 days to about 30 days.

In some embodiments, the nanoparticles have an average diameter of about 1000 nm or less for at least about 15 minutes after nanoparticle formation. In some embodiments, the nanoparticles have an average diameter of about 950 nm or less for at least about 15 minutes after nanoparticle formation. In some embodiments, the nanoparticles have an average diameter of about 900 nm or less for at least about 15 minutes after nanoparticle formation. In some embodiments, the nanoparticles have an average diameter of about 850 nm or less for at least about 15 minutes after nanoparticle formation. In some embodiments, the nanoparticles have an average diameter of about 800 nm or less for at least about 15 minutes after nanoparticle formation. In some embodiments, the nanoparticles have an average diameter of about 750 nm or less for at least about 15 minutes after nanoparticle formation. In some embodiments, the nanoparticles have an average diameter of about 700 nm or less for at least about 15 minutes after nanoparticle formation. In some embodiments, the nanoparticles have an average diameter of about 650 nm or less for at least about 15 minutes after nanoparticle formation. In some embodiments, the nanoparticles have an average diameter of about 600 nm or less for at least about 15 minutes after nanoparticle formation. In some embodiments, the nanoparticles have an average diameter of about 550 nm or less for at least about 15 minutes after nanoparticle formation. In some embodiments, the nanoparticles have an average diameter of about 500 nm or less for at least about 15 minutes after nanoparticle formation. In some embodiments, the nanoparticles have an average diameter of about 450 nm or less for at least about 15 minutes after nanoparticle formation. In some embodiments, the nanoparticles have an average diameter of about 400 nm or less for at least about 15 minutes after nanoparticle formation. In some embodiments, the nanoparticles have an average diameter of about 350 nm or less for at least about 15 minutes after nanoparticle formation. In some embodiments, the nanoparticles have an average diameter of about 300 nm or less for at least about 15 minutes after nanoparticle formation. In some embodiments, the nanoparticles have an average diameter of about 250 nm or less for at least about 15 minutes after nanoparticle formation. In some embodiments, the nanoparticles have an average diameter of about 240 nm or less for at least about 15 minutes after nanoparticle formation. In some embodiments, the nanoparticles have an average diameter of about 230 nm or less for at least about 15 minutes after nanoparticle formation. In some embodiments, the nanoparticles have an average diameter of about 220 nm or less for at least about 15 minutes after nanoparticle formation. In some embodiments, the nanoparticles have an average diameter of about 210 nm or less for at least about 15 minutes after nanoparticle formation. In some embodiments, the nanoparticles have an average diameter of about 200 nm or less for at least about 15 minutes after nanoparticle formation. In some embodiments, the nanoparticles have an average diameter of about 190 nm or less for at least about 15 minutes after nanoparticle formation. In some embodiments, the nanoparticles have an average diameter of about 180 nm or less for at least about 15 minutes after nanoparticle formation. In some embodiments, the nanoparticles have an average diameter of about 170 nm or less for at least about 15 minutes after nanoparticle formation. In some embodiments, the nanoparticles have an average diameter of about 160 nm or less for at least about 15 minutes after nanoparticle formation. In some embodiments, the nanoparticles have an average diameter of about 150 nm or less for at least about 15 minutes after nanoparticle formation. In some embodiments, the nanoparticles have an average diameter of about 140 nm or less for at least about 15 minutes after nanoparticle formation. In some embodiments, the nanoparticles have an average diameter of about 130 nm or less for at least about 15 minutes after nanoparticle formation. In some embodiments, the nanoparticles have an average diameter of about 120 nm or less for at least about 15 minutes after nanoparticle formation. In some embodiments, the nanoparticles have an average diameter of about 110 nm or less for at least about 15 minutes after nanoparticle formation. In some embodiments, the nanoparticles have an average diameter of about 100 nm or less for at least about 15 minutes after nanoparticle formation. In some embodiments, the nanoparticles have an average diameter of about 90 nm or less for at least about 15 minutes after nanoparticle formation. In some embodiments, the nanoparticles have an average diameter of about 80 nm or less for at least about 15 minutes after nanoparticle formation. In some embodiments, the nanoparticles have an average diameter of about 70 nm or less for at least about 15 minutes after nanoparticle formation. In some embodiments, the nanoparticles have an average diameter of about 60 nm or less for at least about 15 minutes after nanoparticle formation. In some embodiments, the nanoparticles have an average diameter of about 50 nm or less for at least about 15 minutes after nanoparticle formation. In some embodiments, the nanoparticles have an average diameter of about 40 nm or less for at least about 15 minutes after nanoparticle formation. In some embodiments, the nanoparticles have an average diameter of about 30 nm or less for at least about 15 minutes after nanoparticle formation. In some embodiments, the nanoparticles have an average diameter of about 20 nm or less for at least about 15 minutes after nanoparticle formation. In some embodiments, the nanoparticles have an average diameter of about 10 nm or less for at least about 15 minutes after nanoparticle formation.

In some embodiments, the nanoparticles have an average diameter of about 10 nm or greater for at least about 15 minutes after nanoparticle formation. In some embodiments, the nanoparticles have an average diameter of about 20 nm or greater for at least about 15 minutes after nanoparticle formation. In some embodiments, the nanoparticles have an average diameter of about 30 nm or greater for at least about 15 minutes after nanoparticle formation. In some embodiments, the nanoparticles have an average diameter of about 40 nm or greater for at least about 15 minutes after nanoparticle formation. In some embodiments, the nanoparticles have an average diameter of about 50 nm or greater for at least about 15 minutes after nanoparticle formation. In some embodiments, the nanoparticles have an average diameter of about 60 nm or greater for at least about 15 minutes after nanoparticle formation. In some embodiments, the nanoparticles have an average diameter of about 70 nm or greater for at least about 15 minutes after nanoparticle formation. In some embodiments, the nanoparticles have an average diameter of about 80 nm or greater for at least about 15 minutes after nanoparticle formation. In some embodiments, the nanoparticles have an average diameter of about 90 nm or greater for at least about 15 minutes after nanoparticle formation. In some embodiments, the nanoparticles have an average diameter of about 100 nm or greater for at least about 15 minutes after nanoparticle formation. In some embodiments, the nanoparticles have an average diameter of about 110 nm or greater for at least about 15 minutes after nanoparticle formation. In some embodiments, the nanoparticles have an average diameter of about 120 nm or greater for at least about 15 minutes after nanoparticle formation. In some embodiments, the nanoparticles have an average diameter of about 130 nm or greater for at least about 15 minutes after nanoparticle formation. In some embodiments, the nanoparticles have an average diameter of about 140 nm or greater for at least about 15 minutes after nanoparticle formation. In some embodiments, the nanoparticles have an average diameter of about 150 nm or greater for at least about 15 minutes after nanoparticle formation. In some embodiments, the nanoparticles have an average diameter of about 160 nm or greater for at least about 15 minutes after nanoparticle formation. In some embodiments, the nanoparticles have an average diameter of about 170 nm or greater for at least about 15 minutes after nanoparticle formation. In some embodiments, the nanoparticles have an average diameter of about 180 nm or greater for at least about 15 minutes after nanoparticle formation. In some embodiments, the nanoparticles have an average diameter of about 190 nm or greater for at least about 15 minutes after nanoparticle formation. In some embodiments, the nanoparticles have an average diameter of about 200 nm or greater for at least about 15 minutes after nanoparticle formation. In some embodiments, the nanoparticles have an average diameter of about 210 nm or greater for at least about 15 minutes after nanoparticle formation. In some embodiments, the nanoparticles have an average diameter of about 220 nm or greater for at least about 15 minutes after nanoparticle formation. In some embodiments, the nanoparticles have an average diameter of about 230 nm or greater for at least about 15 minutes after nanoparticle formation. In some embodiments, the nanoparticles have an average diameter of about 240 nm or greater for at least about 15 minutes after nanoparticle formation. In some embodiments, the nanoparticles have an average diameter of about 250 nm or greater for at least about 15 minutes after nanoparticle formation. In some embodiments, the nanoparticles have an average diameter of about 300 nm or greater for at least about 15 minutes after nanoparticle formation. In some embodiments, the nanoparticles have an average diameter of about 350 nm or greater for at least about 15 minutes after nanoparticle formation. In some embodiments, the nanoparticles have an average diameter of about 400 nm or greater for at least about 15 minutes after nanoparticle formation. In some embodiments, the nanoparticles have an average diameter of about 450 nm or greater for at least about 15 minutes after nanoparticle formation. In some embodiments, the nanoparticles have an average diameter of about 500 nm or greater for at least about 15 minutes after nanoparticle formation. In some embodiments, the nanoparticles have an average diameter of about 550 nm or greater for at least about 15 minutes after nanoparticle formation. In some embodiments, the nanoparticles have an average diameter of about 600 nm or greater for at least about 15 minutes after nanoparticle formation. In some embodiments, the nanoparticles have an average diameter of about 650 nm or greater for at least about 15 minutes after nanoparticle formation. In some embodiments, the nanoparticles have an average diameter of about 700 nm or greater for at least about 15 minutes after nanoparticle formation. In some embodiments, the nanoparticles have an average diameter of about 750 nm or greater for at least about 15 minutes after nanoparticle formation. In some embodiments, the nanoparticles have an average diameter of about 800 nm or greater for at least about 15 minutes after nanoparticle formation. In some embodiments, the nanoparticles have an average diameter of about 850 nm or greater for at least about 15 minutes after nanoparticle formation. In some embodiments, the nanoparticles have an average diameter of about 900 nm or greater for at least about 15 minutes after nanoparticle formation. In some embodiments, the nanoparticles have an average diameter of about 950 nm or greater for at least about 15 minutes after nanoparticle formation

In some embodiments, the nanoparticles have an average diameter of from about 10 nm to about 1000 nm for at least about 15 minutes after nanoparticle formation. In some embodiments, the nanoparticles have an average diameter of from about 10 nm to about 950 nm for at least about 15 minutes after nanoparticle formation. In some embodiments, the nanoparticles have an average diameter of from about 10 nm to about 900 nm for at least about 15 minutes after nanoparticle formation. In some embodiments, the nanoparticles have an average diameter of from about 10 nm to about 850 nm for at least about 15 minutes after nanoparticle formation. In some embodiments, the nanoparticles have an average diameter of from about 10 nm to about 800 nm for at least about 15 minutes after nanoparticle formation. In some embodiments, the nanoparticles have an average diameter of from about 10 nm to about 750 nm for at least about 15 minutes after nanoparticle formation. In some embodiments, the nanoparticles have an average diameter of from about 10 nm to about 700 nm for at least about 15 minutes after nanoparticle formation. In some embodiments, the nanoparticles have an average diameter of from about 10 nm to about 650 nm for at least about 15 minutes after nanoparticle formation. In some embodiments, the nanoparticles have an average diameter of from about 10 nm to about 600 nm for at least about 15 minutes after nanoparticle formation. In some embodiments, the nanoparticles have an average diameter of from about 10 nm to about 550 nm for at least about 15 minutes after nanoparticle formation for at least about 15 minutes after nanoparticle formation. In some embodiments, the nanoparticles have an average diameter of from about 10 nm to about 500 nm for at least about 15 minutes after nanoparticle formation. In some embodiments, the nanoparticles have an average diameter of from about 10 nm to about 450 nm for at least about 15 minutes after nanoparticle formation. In some embodiments, the nanoparticles have an average diameter of from about 10 nm to about 400 nm for at least about 15 minutes after nanoparticle formation. In some embodiments, the nanoparticles have an average diameter of from about 10 nm to about 350 nm for at least about 15 minutes after nanoparticle formation. In some embodiments, the nanoparticles have an average diameter of from about 10 nm to about 300 nm for at least about 15 minutes after nanoparticle formation. In some embodiments, the nanoparticles have an average diameter of from about 10 nm to about 250 nm for at least about 15 minutes after nanoparticle formation. In some embodiments, the nanoparticles have an average diameter of from about 10 nm to about 240 nm for at least about 15 minutes after nanoparticle formation. In some embodiments, the nanoparticles have an average diameter of from about 10 nm to about 230 nm for at least about 15 minutes after nanoparticle formation. In some embodiments, the nanoparticles have an average diameter of from about 10 nm to about 220 nm for at least about 15 minutes after nanoparticle formation. In some embodiments, the nanoparticles have an average diameter of from about 10 nm to about 210 nm for at least about 15 minutes after nanoparticle formation. In some embodiments, the nanoparticles have an average diameter of from about 10 nm to about 200 nm for at least about 15 minutes after nanoparticle formation. In some embodiments, the nanoparticles have an average diameter of from about 10 nm to about 190 nm for at least about 15 minutes after nanoparticle formation. In some embodiments, the nanoparticles have an average diameter of from about 10 nm to about 180 nm for at least about 15 minutes after nanoparticle formation. In some embodiments, the nanoparticles have an average diameter of from about 10 nm to about 170 nm for at least about 15 minutes after nanoparticle formation. In some embodiments, the nanoparticles have an average diameter of from about 10 nm to about 160 nm for at least about 15 minutes after nanoparticle formation. In some embodiments, the nanoparticles have an average diameter of from about 10 nm to about 150 nm for at least about 15 minutes after nanoparticle formation. In some embodiments, the nanoparticles have an average diameter of from about 10 nm to about 140 nm for at least about 15 minutes after nanoparticle formation. In some embodiments, the nanoparticles have an average diameter of from about 10 nm to about 130 nm for at least about 15 minutes after nanoparticle formation. In some embodiments, the nanoparticles have an average diameter of from about 10 nm to about 120 nm for at least about 15 minutes after nanoparticle formation. In some embodiments, the nanoparticles have an average diameter of from about 10 nm to about 110 nm for at least about 15 minutes after nanoparticle formation. In some embodiments, the nanoparticles have an average diameter of from about 10 nm to about 100 nm for at least about 15 minutes after nanoparticle formation. In some embodiments, the nanoparticles have an average diameter of from about 10 nm to about 90 nm for at least about 15 minutes after nanoparticle formation. In some embodiments, the nanoparticles have an average diameter of from about 10 nm to about 80 nm for at least about 15 minutes after nanoparticle formation. In some embodiments, the nanoparticles have an average diameter of from about 10 nm to about 70 nm for at least about 15 minutes after nanoparticle formation. In some embodiments, the nanoparticles have an average diameter of from about 10 nm to about 60 nm for at least about 15 minutes after nanoparticle formation. In some embodiments, the nanoparticles have an average diameter of from about 10 nm to about 50 nm for at least about 15 minutes after nanoparticle formation. In some embodiments, the nanoparticles have an average diameter of from about 10 nm to about 40 nm for at least about 15 minutes after nanoparticle formation. In some embodiments, the nanoparticles have an average diameter of from about 10 nm to about 30 nm for at least about 15 minutes after nanoparticle formation. In some embodiments, the nanoparticles have an average diameter of from about 10 nm to about 20 nm for at least about 15 minutes after nanoparticle formation.

In some embodiments, the nanoparticles have an average diameter of about 10 nm for at least about 15 minutes after nanoparticle formation. In some embodiments, the nanoparticles have an average diameter of about 20 nm for at least about 15 minutes after nanoparticle formation. In some embodiments, the nanoparticles have an average diameter of about 30 nm for at least about 15 minutes after nanoparticle formation. In some embodiments, the nanoparticles have an average diameter of about 40 nm for at least about 15 minutes after nanoparticle formation. In some embodiments, the nanoparticles have an average diameter of about 50 nm for at least about 15 minutes after nanoparticle formation. In some embodiments, the nanoparticles have an average diameter of about 60 nm for at least about 15 minutes after nanoparticle formation. In some embodiments, the nanoparticles have an average diameter of about 70 nm for at least about 15 minutes after nanoparticle formation. In some embodiments, the nanoparticles have an average diameter of about 80 nm for at least about 15 minutes after nanoparticle formation. In some embodiments, the nanoparticles have an average diameter of about 90 nm for at least about 15 minutes after nanoparticle formation. In some embodiments, the nanoparticles have an average diameter of about 100 nm for at least about 15 minutes after nanoparticle formation. In some embodiments, the nanoparticles have an average diameter of about 110 nm for at least about 15 minutes after nanoparticle formation. In some embodiments, the nanoparticles have an average diameter of about 120 nm for at least about 15 minutes after nanoparticle formation. In some embodiments, the nanoparticles have an average diameter of about 130 nm for at least about 15 minutes after nanoparticle formation. In some embodiments, the nanoparticles have an average diameter of about 140 nm for at least about 15 minutes after nanoparticle formation. In some embodiments, the nanoparticles have an average diameter of about 150 nm for at least about 15 minutes after nanoparticle formation. In some embodiments, the nanoparticles have an average diameter of about 160 nm for at least about 15 minutes after nanoparticle formation. In some embodiments, the nanoparticles have an average diameter of about 170 nm for at least about 15 minutes after nanoparticle formation. In some embodiments, the nanoparticles have an average diameter of about 180 nm for at least about 15 minutes after nanoparticle formation. In some embodiments, the nanoparticles have an average diameter of about 190 nm for at least about 15 minutes after nanoparticle formation. In some embodiments, the nanoparticles have an average diameter of about 200 nm for at least about 15 minutes after nanoparticle formation. In some embodiments, the nanoparticles have an average diameter of about 210 nm for at least about 15 minutes after nanoparticle formation. In some embodiments, the nanoparticles have an average diameter of about 220 nm for at least about 15 minutes after nanoparticle formation. In some embodiments, the nanoparticles have an average diameter of about 230 nm for at least about 15 minutes after nanoparticle formation. In some embodiments, the nanoparticles have an average diameter of about 240 nm for at least about 15 minutes after nanoparticle formation. In some embodiments, the nanoparticles have an average diameter of about 250 nm for at least about 15 minutes after nanoparticle formation. In some embodiments, the nanoparticles have an average diameter of about 300 nm for at least about 15 minutes after nanoparticle formation. In some embodiments, the nanoparticles have an average diameter of about 350 nm for at least about 15 minutes after nanoparticle formation. In some embodiments, the nanoparticles have an average diameter of about 400 nm for at least about 15 minutes after nanoparticle formation. In some embodiments, the nanoparticles have an average diameter of about 450 nm for at least about 15 minutes after nanoparticle formation. In some embodiments, the nanoparticles have an average diameter of about 500 nm for at least about 15 minutes after nanoparticle formation. In some embodiments, the nanoparticles have an average diameter of about 550 nm for at least about 15 minutes after nanoparticle formation. In some embodiments, the nanoparticles have an average diameter of about 600 nm for at least about 15 minutes after nanoparticle formation. In some embodiments, the nanoparticles have an average diameter of about 650 nm for at least about 15 minutes after nanoparticle formation. In some embodiments, the nanoparticles have an average diameter of about 700 nm for at least about 15 minutes after nanoparticle formation. In some embodiments, the nanoparticles have an average diameter of about 750 nm for at least about 15 minutes after nanoparticle formation. In some embodiments, the nanoparticles have an average diameter of about 800 nm for at least about 15 minutes after nanoparticle formation. In some embodiments, the nanoparticles have an average diameter of about 850 nm for at least about 15 minutes after nanoparticle formation. In some embodiments, the nanoparticles have an average diameter of about 900 nm for at least about 15 minutes after nanoparticle formation. In some embodiments, the nanoparticles have an average diameter of about 950 nm for at least about 15 minutes after nanoparticle formation. In some embodiments, the nanoparticles have an average diameter of about 1000 nm for at least about 15 minutes after nanoparticle formation.

In some embodiments, the nanoparticles have an average diameter of about 1000 nm or less for at least about 2 hours after nanoparticle formation. In some embodiments, the nanoparticles have an average diameter of about 950 nm or less for at least about 2 hours after nanoparticle formation. In some embodiments, the nanoparticles have an average diameter of about 900 nm or less for at least about 2 hours after nanoparticle formation. In some embodiments, the nanoparticles have an average diameter of about 850 nm or less for at least about 2 hours after nanoparticle formation. In some embodiments, the nanoparticles have an average diameter of about 800 nm or less for at least about 2 hours after nanoparticle formation. In some embodiments, the nanoparticles have an average diameter of about 750 nm or less for at least about 2 hours after nanoparticle formation. In some embodiments, the nanoparticles have an average diameter of about 700 nm or less for at least about 2 hours after nanoparticle formation. In some embodiments, the nanoparticles have an average diameter of about 650 nm or less for at least about 2 hours after nanoparticle formation. In some embodiments, the nanoparticles have an average diameter of about 600 nm or less for at least about 2 hours after nanoparticle formation. In some embodiments, the nanoparticles have an average diameter of about 550 nm or less for at least about 2 hours after nanoparticle formation. In some embodiments, the nanoparticles have an average diameter of about 500 nm or less for at least about 2 hours after nanoparticle formation. In some embodiments, the nanoparticles have an average diameter of about 450 nm or less for at least about 2 hours after nanoparticle formation. In some embodiments, the nanoparticles have an average diameter of about 400 nm or less for at least about 2 hours after nanoparticle formation. In some embodiments, the nanoparticles have an average diameter of about 350 nm or less for at least about 2 hours after nanoparticle formation. In some embodiments, the nanoparticles have an average diameter of about 300 nm or less for at least about 2 hours after nanoparticle formation. In some embodiments, the nanoparticles have an average diameter of about 250 nm or less for at least about 2 hours after nanoparticle formation. In some embodiments, the nanoparticles have an average diameter of about 240 nm or less for at least about 2 hours after nanoparticle formation. In some embodiments, the nanoparticles have an average diameter of about 230 nm or less for at least about 2 hours after nanoparticle formation. In some embodiments, the nanoparticles have an average diameter of about 220 nm or less for at least about 2 hours after nanoparticle formation. In some embodiments, the nanoparticles have an average diameter of about 210 nm or less for at least about 2 hours after nanoparticle formation. In some embodiments, the nanoparticles have an average diameter of about 200 nm or less for at least about 2 hours after nanoparticle formation. In some embodiments, the nanoparticles have an average diameter of about 190 nm or less for at least about 2 hours after nanoparticle formation. In some embodiments, the nanoparticles have an average diameter of about 180 nm or less for at least about 2 hours after nanoparticle formation. In some embodiments, the nanoparticles have an average diameter of about 170 nm or less for at least about 2 hours after nanoparticle formation. In some embodiments, the nanoparticles have an average diameter of about 160 nm or less for at least about 2 hours after nanoparticle formation. In some embodiments, the nanoparticles have an average diameter of about 150 nm or less for at least about 2 hours after nanoparticle formation. In some embodiments, the nanoparticles have an average diameter of about 140 nm or less for at least about 2 hours after nanoparticle formation. In some embodiments, the nanoparticles have an average diameter of about 130 nm or less for at least about 2 hours after nanoparticle formation. In some embodiments, the nanoparticles have an average diameter of about 120 nm or less for at least about 2 hours after nanoparticle formation. In some embodiments, the nanoparticles have an average diameter of about 110 nm or less for at least about 2 hours after nanoparticle formation. In some embodiments, the nanoparticles have an average diameter of about 100 nm or less for at least about 2 hours after nanoparticle formation. In some embodiments, the nanoparticles have an average diameter of about 90 nm or less for at least about 2 hours after nanoparticle formation. In some embodiments, the nanoparticles have an average diameter of about 80 nm or less for at least about 2 hours after nanoparticle formation. In some embodiments, the nanoparticles have an average diameter of about 70 nm or less for at least about 2 hours after nanoparticle formation. In some embodiments, the nanoparticles have an average diameter of about 60 nm or less for at least about 2 hours after nanoparticle formation. In some embodiments, the nanoparticles have an average diameter of about 50 nm or less for at least about 2 hours after nanoparticle formation. In some embodiments, the nanoparticles have an average diameter of about 40 nm or less for at least about 2 hours after nanoparticle formation. In some embodiments, the nanoparticles have an average diameter of about 30 nm or less for at least about 2 hours after nanoparticle formation. In some embodiments, the nanoparticles have an average diameter of about 20 nm or less for at least about 2 hours after nanoparticle formation. In some embodiments, the nanoparticles have an average diameter of about 10 nm or less for at least about 2 hours after nanoparticle formation.

In some embodiments, the nanoparticles have an average diameter of about 10 nm or greater for at least about 2 hours after nanoparticle formation. In some embodiments, the nanoparticles have an average diameter of about 20 nm or greater for at least about 2 hours after nanoparticle formation. In some embodiments, the nanoparticles have an average diameter of about 30 nm or greater for at least about 2 hours after nanoparticle formation. In some embodiments, the nanoparticles have an average diameter of about 40 nm or greater for at least about 2 hours after nanoparticle formation. In some embodiments, the nanoparticles have an average diameter of about 50 nm or greater for at least about 2 hours after nanoparticle formation. In some embodiments, the nanoparticles have an average diameter of about 60 nm or greater for at least about 2 hours after nanoparticle formation. In some embodiments, the nanoparticles have an average diameter of about 70 nm or greater for at least about 2 hours after nanoparticle formation. In some embodiments, the nanoparticles have an average diameter of about 80 nm or greater for at least about 2 hours after nanoparticle formation. In some embodiments, the nanoparticles have an average diameter of about 90 nm or greater for at least about 2 hours after nanoparticle formation. In some embodiments, the nanoparticles have an average diameter of about 100 nm or greater for at least about 2 hours after nanoparticle formation. In some embodiments, the nanoparticles have an average diameter of about 110 nm or greater for at least about 2 hours after nanoparticle formation. In some embodiments, the nanoparticles have an average diameter of about 120 nm or greater for at least about 2 hours after nanoparticle formation. In some embodiments, the nanoparticles have an average diameter of about 130 nm or greater for at least about 2 hours after nanoparticle formation. In some embodiments, the nanoparticles have an average diameter of about 140 nm or greater for at least about 2 hours after nanoparticle formation. In some embodiments, the nanoparticles have an average diameter of about 150 nm or greater for at least about 2 hours after nanoparticle formation. In some embodiments, the nanoparticles have an average diameter of about 160 nm or greater for at least about 2 hours after nanoparticle formation. In some embodiments, the nanoparticles have an average diameter of about 170 nm or greater for at least about 2 hours after nanoparticle formation. In some embodiments, the nanoparticles have an average diameter of about 180 nm or greater for at least about 2 hours after nanoparticle formation. In some embodiments, the nanoparticles have an average diameter of about 190 nm or greater for at least about 2 hours after nanoparticle formation. In some embodiments, the nanoparticles have an average diameter of about 200 nm or greater for at least about 2 hours after nanoparticle formation. In some embodiments, the nanoparticles have an average diameter of about 210 nm or greater for at least about 2 hours after nanoparticle formation. In some embodiments, the nanoparticles have an average diameter of about 220 nm or greater for at least about 2 hours after nanoparticle formation. In some embodiments, the nanoparticles have an average diameter of about 230 nm or greater for at least about 2 hours after nanoparticle formation. In some embodiments, the nanoparticles have an average diameter of about 240 nm or greater for at least about 2 hours after nanoparticle formation. In some embodiments, the nanoparticles have an average diameter of about 250 nm or greater for at least about 2 hours after nanoparticle formation. In some embodiments, the nanoparticles have an average diameter of about 300 nm or greater for at least about 2 hours after nanoparticle formation. In some embodiments, the nanoparticles have an average diameter of about 350 nm or greater for at least about 2 hours after nanoparticle formation. In some embodiments, the nanoparticles have an average diameter of about 400 nm or greater for at least about 2 hours after nanoparticle formation. In some embodiments, the nanoparticles have an average diameter of about 450 nm or greater for at least about 2 hours after nanoparticle formation. In some embodiments, the nanoparticles have an average diameter of about 500 nm or greater for at least about 2 hours after nanoparticle formation. In some embodiments, the nanoparticles have an average diameter of about 550 nm or greater for at least about 2 hours after nanoparticle formation. In some embodiments, the nanoparticles have an average diameter of about 600 nm or greater for at least about 2 hours after nanoparticle formation. In some embodiments, the nanoparticles have an average diameter of about 650 nm or greater for at least about 2 hours after nanoparticle formation. In some embodiments, the nanoparticles have an average diameter of about 700 nm or greater for at least about 2 hours after nanoparticle formation. In some embodiments, the nanoparticles have an average diameter of about 750 nm or greater for at least about 2 hours after nanoparticle formation. In some embodiments, the nanoparticles have an average diameter of about 800 nm or greater for at least about 2 hours after nanoparticle formation. In some embodiments, the nanoparticles have an average diameter of about 850 nm or greater for at least about 2 hours after nanoparticle formation. In some embodiments, the nanoparticles have an average diameter of about 900 nm or greater for at least about 2 hours after nanoparticle formation. In some embodiments, the nanoparticles have an average diameter of about 950 nm or greater for at least about 2 hours after nanoparticle formation

In some embodiments, the nanoparticles have an average diameter of from about 10 nm to about 1000 nm for at least about 2 hours after nanoparticle formation. In some embodiments, the nanoparticles have an average diameter of from about 10 nm to about 950 nm for at least about 2 hours after nanoparticle formation. In some embodiments, the nanoparticles have an average diameter of from about 10 nm to about 900 nm for at least about 2 hours after nanoparticle formation. In some embodiments, the nanoparticles have an average diameter of from about 10 nm to about 850 nm for at least about 2 hours after nanoparticle formation. In some embodiments, the nanoparticles have an average diameter of from about 10 nm to about 800 nm for at least about 2 hours after nanoparticle formation. In some embodiments, the nanoparticles have an average diameter of from about 10 nm to about 750 nm for at least about 2 hours after nanoparticle formation. In some embodiments, the nanoparticles have an average diameter of from about 10 nm to about 700 nm for at least about 2 hours after nanoparticle formation. In some embodiments, the nanoparticles have an average diameter of from about 10 nm to about 650 nm for at least about 2 hours after nanoparticle formation. In some embodiments, the nanoparticles have an average diameter of from about 10 nm to about 600 nm for at least about 2 hours after nanoparticle formation. In some embodiments, the nanoparticles have an average diameter of from about 10 nm to about 550 nm for at least about 2 hours after nanoparticle formation for at least about 2 hours after nanoparticle formation. In some embodiments, the nanoparticles have an average diameter of from about 10 nm to about 500 nm for at least about 2 hours after nanoparticle formation. In some embodiments, the nanoparticles have an average diameter of from about 10 nm to about 450 nm for at least about 2 hours after nanoparticle formation. In some embodiments, the nanoparticles have an average diameter of from about 10 nm to about 400 nm for at least about 2 hours after nanoparticle formation. In some embodiments, the nanoparticles have an average diameter of from about 10 nm to about 350 nm for at least about 2 hours after nanoparticle formation. In some embodiments, the nanoparticles have an average diameter of from about 10 nm to about 300 nm for at least about 2 hours after nanoparticle formation. In some embodiments, the nanoparticles have an average diameter of from about 10 nm to about 250 nm for at least about 2 hours after nanoparticle formation. In some embodiments, the nanoparticles have an average diameter of from about 10 nm to about 240 nm for at least about 2 hours after nanoparticle formation. In some embodiments, the nanoparticles have an average diameter of from about 10 nm to about 230 nm for at least about 2 hours after nanoparticle formation. In some embodiments, the nanoparticles have an average diameter of from about 10 nm to about 220 nm for at least about 2 hours after nanoparticle formation. In some embodiments, the nanoparticles have an average diameter of from about 10 nm to about 210 nm for at least about 2 hours after nanoparticle formation. In some embodiments, the nanoparticles have an average diameter of from about 10 nm to about 200 nm for at least about 2 hours after nanoparticle formation. In some embodiments, the nanoparticles have an average diameter of from about 10 nm to about 190 nm for at least about 2 hours after nanoparticle formation. In some embodiments, the nanoparticles have an average diameter of from about 10 nm to about 180 nm for at least about 2 hours after nanoparticle formation. In some embodiments, the nanoparticles have an average diameter of from about 10 nm to about 170 nm for at least about 2 hours after nanoparticle formation. In some embodiments, the nanoparticles have an average diameter of from about 10 nm to about 160 nm for at least about 2 hours after nanoparticle formation. In some embodiments, the nanoparticles have an average diameter of from about 10 nm to about 150 nm for at least about 2 hours after nanoparticle formation. In some embodiments, the nanoparticles have an average diameter of from about 10 nm to about 140 nm for at least about 2 hours after nanoparticle formation. In some embodiments, the nanoparticles have an average diameter of from about 10 nm to about 130 nm for at least about 2 hours after nanoparticle formation. In some embodiments, the nanoparticles have an average diameter of from about 10 nm to about 120 nm for at least about 2 hours after nanoparticle formation. In some embodiments, the nanoparticles have an average diameter of from about 10 nm to about 110 nm for at least about 2 hours after nanoparticle formation. In some embodiments, the nanoparticles have an average diameter of from about 10 nm to about 100 nm for at least about 2 hours after nanoparticle formation. In some embodiments, the nanoparticles have an average diameter of from about 10 nm to about 90 nm for at least about 2 hours after nanoparticle formation. In some embodiments, the nanoparticles have an average diameter of from about 10 nm to about 80 nm for at least about 2 hours after nanoparticle formation. In some embodiments, the nanoparticles have an average diameter of from about 10 nm to about 70 nm for at least about 2 hours after nanoparticle formation. In some embodiments, the nanoparticles have an average diameter of from about 10 nm to about 60 nm for at least about 2 hours after nanoparticle formation. In some embodiments, the nanoparticles have an average diameter of from about 10 nm to about 50 nm for at least about 2 hours after nanoparticle formation. In some embodiments, the nanoparticles have an average diameter of from about 10 nm to about 40 nm for at least about 2 hours after nanoparticle formation. In some embodiments, the nanoparticles have an average diameter of from about 10 nm to about 30 nm for at least about 2 hours after nanoparticle formation. In some embodiments, the nanoparticles have an average diameter of from about 10 nm to about 20 nm for at least about 2 hours after nanoparticle formation.

In some embodiments, the nanoparticles have an average diameter of about 10 nm for at least about 2 hours after nanoparticle formation. In some embodiments, the nanoparticles have an average diameter of about 20 nm for at least about 2 hours after nanoparticle formation. In some embodiments, the nanoparticles have an average diameter of about 30 nm for at least about 2 hours after nanoparticle formation. In some embodiments, the nanoparticles have an average diameter of about 40 nm for at least about 2 hours after nanoparticle formation. In some embodiments, the nanoparticles have an average diameter of about 50 nm for at least about 2 hours after nanoparticle formation. In some embodiments, the nanoparticles have an average diameter of about 60 nm for at least about 2 hours after nanoparticle formation. In some embodiments, the nanoparticles have an average diameter of about 70 nm for at least about 2 hours after nanoparticle formation. In some embodiments, the nanoparticles have an average diameter of about 80 nm for at least about 2 hours after nanoparticle formation. In some embodiments, the nanoparticles have an average diameter of about 90 nm for at least about 2 hours after nanoparticle formation. In some embodiments, the nanoparticles have an average diameter of about 100 nm for at least about 2 hours after nanoparticle formation. In some embodiments, the nanoparticles have an average diameter of about 110 nm for at least about 2 hours after nanoparticle formation. In some embodiments, the nanoparticles have an average diameter of about 120 nm for at least about 2 hours after nanoparticle formation. In some embodiments, the nanoparticles have an average diameter of about 130 nm for at least about 2 hours after nanoparticle formation. In some embodiments, the nanoparticles have an average diameter of about 140 nm for at least about 2 hours after nanoparticle formation. In some embodiments, the nanoparticles have an average diameter of about 150 nm for at least about 2 hours after nanoparticle formation. In some embodiments, the nanoparticles have an average diameter of about 160 nm for at least about 2 hours after nanoparticle formation. In some embodiments, the nanoparticles have an average diameter of about 170 nm for at least about 2 hours after nanoparticle formation. In some embodiments, the nanoparticles have an average diameter of about 180 nm for at least about 2 hours after nanoparticle formation. In some embodiments, the nanoparticles have an average diameter of about 190 nm for at least about 2 hours after nanoparticle formation. In some embodiments, the nanoparticles have an average diameter of about 200 nm for at least about 2 hours after nanoparticle formation. In some embodiments, the nanoparticles have an average diameter of about 210 nm for at least about 2 hours after nanoparticle formation. In some embodiments, the nanoparticles have an average diameter of about 220 nm for at least about 2 hours after nanoparticle formation. In some embodiments, the nanoparticles have an average diameter of about 230 nm for at least about 2 hours after nanoparticle formation. In some embodiments, the nanoparticles have an average diameter of about 240 nm for at least about 2 hours after nanoparticle formation. In some embodiments, the nanoparticles have an average diameter of about 250 nm for at least about 2 hours after nanoparticle formation. In some embodiments, the nanoparticles have an average diameter of about 300 nm for at least about 2 hours after nanoparticle formation. In some embodiments, the nanoparticles have an average diameter of about 350 nm for at least about 2 hours after nanoparticle formation. In some embodiments, the nanoparticles have an average diameter of about 400 nm for at least about 2 hours after nanoparticle formation. In some embodiments, the nanoparticles have an average diameter of about 450 nm for at least about 2 hours after nanoparticle formation. In some embodiments, the nanoparticles have an average diameter of about 500 nm for at least about 2 hours after nanoparticle formation. In some embodiments, the nanoparticles have an average diameter of about 550 nm for at least about 2 hours after nanoparticle formation. In some embodiments, the nanoparticles have an average diameter of about 600 nm for at least about 2 hours after nanoparticle formation. In some embodiments, the nanoparticles have an average diameter of about 650 nm for at least about 2 hours after nanoparticle formation. In some embodiments, the nanoparticles have an average diameter of about 700 nm for at least about 2 hours after nanoparticle formation. In some embodiments, the nanoparticles have an average diameter of about 750 nm for at least about 2 hours after nanoparticle formation. In some embodiments, the nanoparticles have an average diameter of about 800 nm for at least about 2 hours after nanoparticle formation. In some embodiments, the nanoparticles have an average diameter of about 850 nm for at least about 2 hours after nanoparticle formation. In some embodiments, the nanoparticles have an average diameter of about 900 nm for at least about 2 hours after nanoparticle formation. In some embodiments, the nanoparticles have an average diameter of about 950 nm for at least about 2 hours after nanoparticle formation. In some embodiments, the nanoparticles have an average diameter of about 1000 nm for at least about 2 hours after nanoparticle formation.

In some embodiments, the nanoparticles have an average diameter of from about 10 nm to about 1000 nm. In some embodiments, the nanoparticles have an average diameter of from about 10 nm to about 950 nm. In some embodiments, the nanoparticles have an average diameter of from about 10 nm to about 900 nm. In some embodiments, the nanoparticles have an average diameter of from about 10 nm to about 850 nm. In some embodiments, the nanoparticles have an average diameter of from about 10 nm to about 800 nm. In some embodiments, the nanoparticles have an average diameter of from about 10 nm to about 750 nm. In some embodiments, the nanoparticles have an average diameter of from about 10 nm to about 700 nm. In some embodiments, the nanoparticles have an average diameter of from about 10 nm to about 650 nm. In some embodiments, the nanoparticles have an average diameter of from about 10 nm to about 600 nm. In some embodiments, the nanoparticles have an average diameter of from about 10 nm to about 550 nm. In some embodiments, the nanoparticles have an average diameter of from about 10 nm to about 500 nm. In some embodiments, the nanoparticles have an average diameter of from about 10 nm to about 450 nm. In some embodiments, the nanoparticles have an average diameter of from about 10 nm to about 400 nm. In some embodiments, the nanoparticles have an average diameter of from about 10 nm to about 350 nm. In some embodiments, the nanoparticles have an average diameter of from about 10 nm to about 300 nm. In some embodiments, the nanoparticles have an average diameter of from about 10 nm to about 250 nm. In some embodiments, the nanoparticles have an average diameter of from about 10 nm to about 240 nm. In some embodiments, the nanoparticles have an average diameter of from about 10 nm to about 230 nm. In some embodiments, the nanoparticles have an average diameter of from about 10 nm to about 220 nm. In some embodiments, the nanoparticles have an average diameter of from about 10 nm to about 210 nm. In some embodiments, the nanoparticles have an average diameter of from about 10 nm to about 200 nm. In some embodiments, the nanoparticles have an average diameter of from about 10 nm to about 190 nm. In some embodiments, the nanoparticles have an average diameter of from about 10 nm to about 180 nm. In some embodiments, the nanoparticles have an average diameter of from about 10 nm to about 170 nm. In some embodiments, the nanoparticles have an average diameter of from about 10 nm to about 160 nm. In some embodiments, the nanoparticles have an average diameter of from about 10 nm to about 150 nm. In some embodiments, the nanoparticles have an average diameter of from about 10 nm to about 140 nm. In some embodiments, the nanoparticles have an average diameter of from about 10 nm to about 130 nm. In some embodiments, the nanoparticles have an average diameter of from about 10 nm to about 120 nm. In some embodiments, the nanoparticles have an average diameter of from about 10 nm to about 110 nm. In some embodiments, the nanoparticles have an average diameter of from about 10 nm to about 100 nm. In some embodiments, the nanoparticles have an average diameter of from about 10 nm to about 90 nm. In some embodiments, the nanoparticles have an average diameter of from about 10 nm to about 80 nm. In some embodiments, the nanoparticles have an average diameter of from about 10 nm to about 70 nm. In some embodiments, the nanoparticles have an average diameter of from about 10 nm to about 60 nm. In some embodiments, the nanoparticles have an average diameter of from about 10 nm to about 50 nm. In some embodiments, the nanoparticles have an average diameter of from about 10 nm to about 40 nm. In some embodiments, the nanoparticles have an average diameter of from about 10 nm to about 30 nm. In some embodiments, the nanoparticles have an average diameter of from about 10 nm to about 20 nm.

In some embodiments, the nanoparticles have an average diameter of from about 20 nm to about 1000 nm. In some embodiments, the nanoparticles have an average diameter of from about 20 nm to about 950 nm. In some embodiments, the nanoparticles have an average diameter of from about 20 nm to about 900 nm. In some embodiments, the nanoparticles have an average diameter of from about 20 nm to about 850 nm. In some embodiments, the nanoparticles have an average diameter of from about 20 nm to about 800 nm. In some embodiments, the nanoparticles have an average diameter of from about 20 nm to about 750 nm. In some embodiments, the nanoparticles have an average diameter of from about 20 nm to about 700 nm. In some embodiments, the nanoparticles have an average diameter of from about 20 nm to about 650 nm. In some embodiments, the nanoparticles have an average diameter of from about 20 nm to about 600 nm. In some embodiments, the nanoparticles have an average diameter of from about 20 nm to about 550 nm. In some embodiments, the nanoparticles have an average diameter of from about 20 nm to about 500 nm. In some embodiments, the nanoparticles have an average diameter of from about 20 nm to about 450 nm. In some embodiments, the nanoparticles have an average diameter of from about 20 nm to about 400 nm. In some embodiments, the nanoparticles have an average diameter of from about 20 nm to about 350 nm. In some embodiments, the nanoparticles have an average diameter of from about 20 nm to about 300 nm. In some embodiments, the nanoparticles have an average diameter of from about 20 nm to about 250 nm. In some embodiments, the nanoparticles have an average diameter of from about 20 nm to about 240 nm. In some embodiments, the nanoparticles have an average diameter of from about 20 nm to about 230 nm. In some embodiments, the nanoparticles have an average diameter of from about 20 nm to about 220 nm. In some embodiments, the nanoparticles have an average diameter of from about 20 nm to about 210 nm. In some embodiments, the nanoparticles have an average diameter of from about 20 nm to about 200 nm. In some embodiments, the nanoparticles have an average diameter of from about 20 nm to about 190 nm. In some embodiments, the nanoparticles have an average diameter of from about 20 nm to about 180 nm. In some embodiments, the nanoparticles have an average diameter of from about 20 nm to about 170 nm. In some embodiments, the nanoparticles have an average diameter of from about 20 nm to about 160 nm. In some embodiments, the nanoparticles have an average diameter of from about 20 nm to about 150 nm. In some embodiments, the nanoparticles have an average diameter of from about 20 nm to about 140 nm. In some embodiments, the nanoparticles have an average diameter of from about 20 nm to about 130 nm. In some embodiments, the nanoparticles have an average diameter of from about 20 nm to about 120 nm. In some embodiments, the nanoparticles have an average diameter of from about 20 nm to about 110 nm. In some embodiments, the nanoparticles have an average diameter of from about 20 nm to about 100 nm. In some embodiments, the nanoparticles have an average diameter of from about 20 nm to about 90 nm. In some embodiments, the nanoparticles have an average diameter of from about 20 nm to about 80 nm. In some embodiments, the nanoparticles have an average diameter of from about 20 nm to about 70 nm. In some embodiments, the nanoparticles have an average diameter of from about 20 nm to about 60 nm. In some embodiments, the nanoparticles have an average diameter of from about 20 nm to about 50 nm. In some embodiments, the nanoparticles have an average diameter of from about 20 nm to about 40 nm. In some embodiments, the nanoparticles have an average diameter of from about 20 nm to about 30 nm.

In some embodiments, the nanoparticles have an average diameter of from about 30 nm to about 1000 nm. In some embodiments, the nanoparticles have an average diameter of from about 30 nm to about 950 nm. In some embodiments, the nanoparticles have an average diameter of from about 30 nm to about 900 nm. In some embodiments, the nanoparticles have an average diameter of from about 30 nm to about 850 nm. In some embodiments, the nanoparticles have an average diameter of from about 30 nm to about 800 nm. In some embodiments, the nanoparticles have an average diameter of from about 30 nm to about 750 nm. In some embodiments, the nanoparticles have an average diameter of from about 30 nm to about 700 nm. In some embodiments, the nanoparticles have an average diameter of from about 30 nm to about 650 nm. In some embodiments, the nanoparticles have an average diameter of from about 30 nm to about 600 nm. In some embodiments, the nanoparticles have an average diameter of from about 30 nm to about 550 nm. In some embodiments, the nanoparticles have an average diameter of from about 30 nm to about 500 nm. In some embodiments, the nanoparticles have an average diameter of from about 30 nm to about 450 nm. In some embodiments, the nanoparticles have an average diameter of from about 30 nm to about 400 nm. In some embodiments, the nanoparticles have an average diameter of from about 30 nm to about 350 nm. In some embodiments, the nanoparticles have an average diameter of from about 30 nm to about 300 nm. In some embodiments, the nanoparticles have an average diameter of from about 30 nm to about 250 nm. In some embodiments, the nanoparticles have an average diameter of from about 30 nm to about 240 nm. In some embodiments, the nanoparticles have an average diameter of from about 30 nm to about 230 nm. In some embodiments, the nanoparticles have an average diameter of from about 30 nm to about 220 nm. In some embodiments, the nanoparticles have an average diameter of from about 30 nm to about 210 nm. In some embodiments, the nanoparticles have an average diameter of from about 30 nm to about 200 nm. In some embodiments, the nanoparticles have an average diameter of from about 30 nm to about 190 nm. In some embodiments, the nanoparticles have an average diameter of from about 30 nm to about 180 nm. In some embodiments, the nanoparticles have an average diameter of from about 30 nm to about 170 nm. In some embodiments, the nanoparticles have an average diameter of from about 30 nm to about 160 nm. In some embodiments, the nanoparticles have an average diameter of from about 30 nm to about 150 nm. In some embodiments, the nanoparticles have an average diameter of from about 30 nm to about 140 nm. In some embodiments, the nanoparticles have an average diameter of from about 30 nm to about 130 nm. In some embodiments, the nanoparticles have an average diameter of from about 30 nm to about 120 nm. In some embodiments, the nanoparticles have an average diameter of from about 30 nm to about 110 nm. In some embodiments, the nanoparticles have an average diameter of from about 30 nm to about 100 nm. In some embodiments, the nanoparticles have an average diameter of from about 30 nm to about 90 nm. In some embodiments, the nanoparticles have an average diameter of from about 30 nm to about 80 nm. In some embodiments, the nanoparticles have an average diameter of from about 30 nm to about 70 nm. In some embodiments, the nanoparticles have an average diameter of from about 30 nm to about 60 nm. In some embodiments, the nanoparticles have an average diameter of from about 30 nm to about 50 nm. In some embodiments, the nanoparticles have an average diameter of from about 30 nm to about 40 nm. In some embodiments, the nanoparticles have an average diameter of from about 30 nm to about 40 nm.

In some embodiments, the nanoparticles have an average diameter of from about 40 nm to about 1000 nm. In some embodiments, the nanoparticles have an average diameter of from about 40 nm to about 950 nm. In some embodiments, the nanoparticles have an average diameter of from about 40 nm to about 900 nm. In some embodiments, the nanoparticles have an average diameter of from about 40 nm to about 850 nm. In some embodiments, the nanoparticles have an average diameter of from about 40 nm to about 800 nm. In some embodiments, the nanoparticles have an average diameter of from about 40 nm to about 750 nm. In some embodiments, the nanoparticles have an average diameter of from about 40 nm to about 700 nm. In some embodiments, the nanoparticles have an average diameter of from about 40 nm to about 650 nm. In some embodiments, the nanoparticles have an average diameter of from about 40 nm to about 600 nm. In some embodiments, the nanoparticles have an average diameter of from about 40 nm to about 550 nm. In some embodiments, the nanoparticles have an average diameter of from about 40 nm to about 500 nm. In some embodiments, the nanoparticles have an average diameter of from about 40 nm to about 450 nm. In some embodiments, the nanoparticles have an average diameter of from about 40 nm to about 400 nm. In some embodiments, the nanoparticles have an average diameter of from about 40 nm to about 350 nm. In some embodiments, the nanoparticles have an average diameter of from about 40 nm to about 300 nm. In some embodiments, the nanoparticles have an average diameter of from about 40 nm to about 250 nm. In some embodiments, the nanoparticles have an average diameter of from about 40 nm to about 240 nm. In some embodiments, the nanoparticles have an average diameter of from about 40 nm to about 230 nm. In some embodiments, the nanoparticles have an average diameter of from about 40 nm to about 220 nm. In some embodiments, the nanoparticles have an average diameter of from about 40 nm to about 210 nm. In some embodiments, the nanoparticles have an average diameter of from about 40 nm to about 200 nm. In some embodiments, the nanoparticles have an average diameter of from about 40 nm to about 190 nm. In some embodiments, the nanoparticles have an average diameter of from about 40 nm to about 180 nm. In some embodiments, the nanoparticles have an average diameter of from about 40 nm to about 170 nm. In some embodiments, the nanoparticles have an average diameter of from about 40 nm to about 160 nm. In some embodiments, the nanoparticles have an average diameter of from about 40 nm to about 150 nm. In some embodiments, the nanoparticles have an average diameter of from about 40 nm to about 140 nm. In some embodiments, the nanoparticles have an average diameter of from about 40 nm to about 130 nm. In some embodiments, the nanoparticles have an average diameter of from about 40 nm to about 120 nm. In some embodiments, the nanoparticles have an average diameter of from about 40 nm to about 110 nm. In some embodiments, the nanoparticles have an average diameter of from about 40 nm to about 100 nm. In some embodiments, the nanoparticles have an average diameter of from about 40 nm to about 90 nm. In some embodiments, the nanoparticles have an average diameter of from about 40 nm to about 80 nm. In some embodiments, the nanoparticles have an average diameter of from about 40 nm to about 70 nm. In some embodiments, the nanoparticles have an average diameter of from about 40 nm to about 60 nm. In some embodiments, the nanoparticles have an average diameter of from about 40 nm to about 50 nm.

In some embodiments, the nanoparticles have an average diameter of from about 50 nm to about 1000 nm. In some embodiments, the nanoparticles have an average diameter of from about 50 nm to about 950 nm. In some embodiments, the nanoparticles have an average diameter of from about 50 nm to about 900 nm. In some embodiments, the nanoparticles have an average diameter of from about 50 nm to about 850 nm. In some embodiments, the nanoparticles have an average diameter of from about 50 nm to about 800 nm. In some embodiments, the nanoparticles have an average diameter of from about 50 nm to about 750 nm. In some embodiments, the nanoparticles have an average diameter of from about 50 nm to about 700 nm. In some embodiments, the nanoparticles have an average diameter of from about 50 nm to about 650 nm. In some embodiments, the nanoparticles have an average diameter of from about 50 nm to about 600 nm. In some embodiments, the nanoparticles have an average diameter of from about 50 nm to about 550 nm. In some embodiments, the nanoparticles have an average diameter of from about 50 nm to about 500 nm. In some embodiments, the nanoparticles have an average diameter of from about 50 nm to about 450 nm. In some embodiments, the nanoparticles have an average diameter of from about 50 nm to about 400 nm. In some embodiments, the nanoparticles have an average diameter of from about 50 nm to about 350 nm. In some embodiments, the nanoparticles have an average diameter of from about 50 nm to about 300 nm. In some embodiments, the nanoparticles have an average diameter of from about 50 nm to about 250 nm. In some embodiments, the nanoparticles have an average diameter of from about 50 nm to about 240 nm. In some embodiments, the nanoparticles have an average diameter of from about 50 nm to about 230 nm. In some embodiments, the nanoparticles have an average diameter of from about 50 nm to about 220 nm. In some embodiments, the nanoparticles have an average diameter of from about 50 nm to about 210 nm. In some embodiments, the nanoparticles have an average diameter of from about 50 nm to about 200 nm. In some embodiments, the nanoparticles have an average diameter of from about 50 nm to about 190 nm. In some embodiments, the nanoparticles have an average diameter of from about 50 nm to about 180 nm. In some embodiments, the nanoparticles have an average diameter of from about 50 nm to about 170 nm. In some embodiments, the nanoparticles have an average diameter of from about 50 nm to about 160 nm. In some embodiments, the nanoparticles have an average diameter of from about 50 nm to about 150 nm. In some embodiments, the nanoparticles have an average diameter of from about 50 nm to about 140 nm. In some embodiments, the nanoparticles have an average diameter of from about 50 nm to about 130 nm. In some embodiments, the nanoparticles have an average diameter of from about 50 nm to about 120 nm. In some embodiments, the nanoparticles have an average diameter of from about 50 nm to about 110 nm. In some embodiments, the nanoparticles have an average diameter of from about 50 nm to about 100 nm. In some embodiments, the nanoparticles have an average diameter of from about 50 nm to about 90 nm. In some embodiments, the nanoparticles have an average diameter of from about 50 nm to about 80 nm. In some embodiments, the nanoparticles have an average diameter of from about 50 nm to about 70 nm. In some embodiments, the nanoparticles have an average diameter of from about 50 nm to about 60 nm.

In some embodiments, the nanoparticles have an average diameter of about 10 nm. In some embodiments, the nanoparticles have an average diameter of about 20 nm. In some embodiments, the nanoparticles have an average diameter of about 30 nm. In some embodiments, the nanoparticles have an average diameter of about 40 nm. In some embodiments, the nanoparticles have an average diameter of about 50 nm. In some embodiments, the nanoparticles have an average diameter of about 60 nm. In some embodiments, the nanoparticles have an average diameter of about 70 nm. In some embodiments, the nanoparticles have an average diameter of about 80 nm. In some embodiments, the nanoparticles have an average diameter of about 90 nm. In some embodiments, the nanoparticles have an average diameter of about 100 nm. In some embodiments, the nanoparticles have an average diameter of about 110 nm. In some embodiments, the nanoparticles have an average diameter of about 120 nm. In some embodiments, the nanoparticles have an average diameter of about 130 nm. In some embodiments, the nanoparticles have an average diameter of about 140 nm. In some embodiments, the nanoparticles have an average diameter of about 150 nm. In some embodiments, the nanoparticles have an average diameter of about 160 nm. In some embodiments, the nanoparticles have an average diameter of about 170 nm. In some embodiments, the nanoparticles have an average diameter of about 180 nm. In some embodiments, the nanoparticles have an average diameter of about 190 nm. In some embodiments, the nanoparticles have an average diameter of about 200 nm. In some embodiments, the nanoparticles have an average diameter of about 210 nm. In some embodiments, the nanoparticles have an average diameter of about 220 nm. In some embodiments, the nanoparticles have an average diameter of about 230 nm. In some embodiments, the nanoparticles have an average diameter of about 240 nm. In some embodiments, the nanoparticles have an average diameter of about 250 nm. In some embodiments, the nanoparticles have an average diameter of about 300 nm. In some embodiments, the nanoparticles have an average diameter of about 350 nm. In some embodiments, the nanoparticles have an average diameter of about 400 nm. In some embodiments, the nanoparticles have an average diameter of about 450 nm. In some embodiments, the nanoparticles have an average diameter of about 500 nm. In some embodiments, the nanoparticles have an average diameter of about 550 nm. In some embodiments, the nanoparticles have an average diameter of about 600 nm. In some embodiments, the nanoparticles have an average diameter of about 650 nm. In some embodiments, the nanoparticles have an average diameter of about 700 nm. In some embodiments, the nanoparticles have an average diameter of about 750 nm. In some embodiments, the nanoparticles have an average diameter of about 800 nm. In some embodiments, the nanoparticles have an average diameter of about 850 nm. In some embodiments, the nanoparticles have an average diameter of about 900 nm. In some embodiments, the nanoparticles have an average diameter of about 950 nm. In some embodiments, the nanoparticles have an average diameter of about 1000 nm.

In some embodiments, the composition is sterile filterable. In some embodiments, the nanoparticles have an average diameter of about 250 nm or less. In some embodiments, the nanoparticles have an average diameter of about 240 nm or less. In some embodiments, the nanoparticles have an average diameter of about 230 nm or less. In some embodiments, the nanoparticles have an average diameter of about 220 nm or less. In some embodiments, the nanoparticles have an average diameter of about 210 nm or less. In some embodiments, the nanoparticles have an average diameter of about 200 nm or less. In some embodiments, the nanoparticles have an average diameter of from about 10 nm to about 250 nm. In some embodiments, the nanoparticles have an average diameter of from about 10 nm to about 240 nm. In some embodiments, the nanoparticles have an average diameter of from about 10 nm to about 230 nm. In some embodiments, the nanoparticles have an average diameter of from about 10 nm to about 220 nm. In some embodiments, the nanoparticles have an average diameter of from about 10 nm to about 210 nm. In some embodiments, the nanoparticles have an average diameter of from about 10 nm to about 200 nm.

In some embodiments, the nanoparticles are suspended, dissolved, or emulsified in a liquid. In some embodiments, the nanoparticles are suspended in a liquid. In some embodiments, the nanoparticles are dissolved in a liquid. In some embodiments, the nanoparticles are emulsified in a liquid. In some embodiments, the nanoparticles are cross-linked using glutaraldehyde, glucose, or UV irradiation.

### Dehydrated Composition

In some embodiments, the composition is dehydrated. In some embodiments, the composition is a lyophilized composition. In some embodiments, the dehydrated composition comprises less than about 10%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.05%, or about 0.01% by weight of water. In some embodiments, the dehydrated composition comprises less than about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.05%, or about 0.01% by weight of water.

In some embodiments, when the composition is dehydrated composition, such as a lyophilized composition, the composition comprises from about 0.1% to about 99% by weight of the compound. In some embodiments, the composition comprises from about 0.1% to about 75% by weight of the compound. In some embodiments, the composition comprises from about 0.1% to about 50% by weight of the compound. In some embodiments, the composition comprises from about 0.1% to about 25% by weight of the compound. In some embodiments, the composition comprises from about 0.1% to about 20% by weight of the compound. In some embodiments, the composition comprises from about 0.1% to about 15% by weight of the compound. In some embodiments, the composition comprises from about 0.1% to about 10% by weight of the compound.

In some embodiments, when the composition is dehydrated composition, such as a lyophilized composition, the composition comprises from about 0.5% to about 99% by weight of the compound. In some embodiments, the composition comprises from about 0.5% to about 75% by weight of the compound. In some embodiments, the composition comprises from about 0.5% to about 50% by weight of the compound. In some embodiments, the composition comprises from about 0.5% to about 25% by weight of the compound. In some embodiments, the composition comprises from about 0.5% to about 20% by weight of the compound. In some embodiments, the composition comprises from about 0.5% to about 15% by weight of the compound. In some embodiments, the composition comprises from about 0.5% to about 10% by weight of the compound.

In some embodiments, when the composition is dehydrated composition, such as a lyophilized composition, the composition comprises from about 0.9% to about 24% by weight of the compound. In some embodiments, the composition comprises from about 1.8% to about 16% by weight of the compound.

In some embodiments, when the composition is dehydrated composition, such as a lyophilized composition, the composition comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% by weight of the compound. In some embodiments, the composition comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the compound. In some embodiments, the composition comprises about 0.9%, about 1%, about 1.1%, about 1.2%, about 1.3%, about 1.4%, about 1.5%, about 1.6%, about 1.7%, about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, or about 24% by weight of the compound. In some embodiments, the composition comprises about 1.8%, about 1.9% about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, or about 16% by weight of the compound.

In some embodiments, when the composition is dehydrated composition, such as a lyophilized composition, the composition comprises from about 50% to about 99% by weight of the pharmaceutically acceptable carrier. In some embodiments, the composition comprises from about 55% to about 99% by weight of the pharmaceutically acceptable carrier. In some embodiments, the composition comprises from about 60% to about 99% by weight of the pharmaceutically acceptable carrier. In some embodiments, the composition comprises from about 65% to about 99% by weight of the pharmaceutically acceptable carrier. In some embodiments, the composition comprises from about 70% to about 99% by weight of the pharmaceutically acceptable carrier. In some embodiments, the composition comprises from about 75% to about 99% by weight of the pharmaceutically acceptable carrier. In some embodiments, the composition comprises from about 80% to about 99% by weight of the pharmaceutically acceptable carrier. In some embodiments, the composition comprises from about 85% to about 99% by weight of the pharmaceutically acceptable carrier. In some embodiments, the composition comprises from about 90% to about 99% by weight of the pharmaceutically acceptable carrier.

In some embodiments, when the composition is dehydrated composition, such as a lyophilized composition, the composition comprises from about 76% to about 99% by weight of the pharmaceutically acceptable carrier. In some embodiments, the composition comprises from about 84% to about 98% by weight of the pharmaceutically acceptable carrier.

In some embodiments, when the composition is dehydrated composition, such as a lyophilized composition, the composition comprises about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier. In some embodiments, the composition comprises about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier. In some embodiments, the composition comprises about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the pharmaceutically acceptable carrier.

### Reconstitution

In some embodiments, the composition is reconstituted with an appropriate biocompatible liquid to provide a reconstituted composition. In some embodiments, appropriate biocompatible liquid is a buffered solution. Examples of suitable buffered solutions include, but are not limited to, buffered solutions of amino acids, buffered solutions of proteins, buffered solutions of sugars, buffered solutions of vitamins, buffered solutions of synthetic polymers, buffered solutions of salts (such as buffered saline or buffered aqueous media), any similar buffered solutions, or any suitable combination thereof. In some embodiments, the appropriate biocompatible liquid is a solution comprising dextrose. In some embodiments, the appropriate biocompatible liquid is a solution comprising one or more salts. In some embodiments, the appropriate biocompatible liquid is a solution suitable for intravenous use. Examples of solutions that are suitable for intravenous use, include, but are not limited to, balanced solutions, which are different solutions with different electrolyte compositions that are close to plasma composition. Such electrolyte compositions comprise crystalloids or colloids. Examples of suitable appropriate biocompatible liquids include, but are not limited to, sterile water, saline, phosphate-buffered saline, 5% dextrose in water solution, Ringer's solution, or Ringer's lactate solution. In some embodiments, the appropriate biocompatible liquid is sterile water, saline, phosphate-buffered saline, 5% dextrose in water solution, Ringer's solution, or Ringer's lactate solution. In some embodiments, the appropriate biocompatible liquid is sterile water. In some embodiments, the appropriate biocompatible liquid is saline. In some embodiments, the appropriate biocompatible liquid is phosphate-buffered saline. In some embodiments, the appropriate biocompatible liquid is 5% dextrose in water solution. In some embodiments, the appropriate biocompatible liquid is Ringer's solution. In some embodiments, the appropriate biocompatible liquid is Ringer's lactate solution. In some embodiments, the appropriate biocompatible liquid is a balanced solution, or a solution with an electrolyte composition that resembles plasma.

In some embodiments, the nanoparticles have an average diameter of from about 10 nm to about 1000 nm after reconstitution. In some embodiments, the nanoparticles have an average diameter of from about 10 nm to about 950 nm after reconstitution. In some embodiments, the nanoparticles have an average diameter of from about 10 nm to about 900 nm after reconstitution. In some embodiments, the nanoparticles have an average diameter of from about 10 nm to about 850 nm after reconstitution. In some embodiments, the nanoparticles have an average diameter of from about 10 nm to about 800 nm after reconstitution. In some embodiments, the nanoparticles have an average diameter of from about 10 nm to about 750 nm after reconstitution. In some embodiments, the nanoparticles have an average diameter of from about 10 nm to about 700 nm after reconstitution. In some embodiments, the nanoparticles have an average diameter of from about 10 nm to about 650 nm after reconstitution. In some embodiments, the nanoparticles have an average diameter of from about 10 nm to about 600 nm after reconstitution. In some embodiments, the nanoparticles have an average diameter of from about 10 nm to about 550 nm after reconstitution. In some embodiments, the nanoparticles have an average diameter of from about 10 nm to about 500 nm after reconstitution. In some embodiments, the nanoparticles have an average diameter of from about 10 nm to about 450 nm after reconstitution. In some embodiments, the nanoparticles have an average diameter of from about 10 nm to about 400 nm after reconstitution. In some embodiments, the nanoparticles have an average diameter of from about 10 nm to about 350 nm after reconstitution. In some embodiments, the nanoparticles have an average diameter of from about 10 nm to about 300 nm after reconstitution. In some embodiments, the nanoparticles have an average diameter of from about 10 nm to about 250 nm after reconstitution. In some embodiments, the nanoparticles have an average diameter of from about 10 nm to about 240 nm after reconstitution. In some embodiments, the nanoparticles have an average diameter of from about 10 nm to about 230 nm after reconstitution. In some embodiments, the nanoparticles have an average diameter of from about 10 nm to about 220 nm after reconstitution. In some embodiments, the nanoparticles have an average diameter of from about 10 nm to about 210 nm after reconstitution. In some embodiments, the nanoparticles have an average diameter of from about 10 nm to about 200 nm after reconstitution. In some embodiments, the nanoparticles have an average diameter of from about 10 nm to about 190 nm after reconstitution. In some embodiments, the nanoparticles have an average diameter of from about 10 nm to about 180 nm after reconstitution. In some embodiments, the nanoparticles have an average diameter of from about 10 nm to about 170 nm after reconstitution. In some embodiments, the nanoparticles have an average diameter of from about 10 nm to about 160 nm after reconstitution. In some embodiments, the nanoparticles have an average diameter of from about 10 nm to about 150 nm after reconstitution. In some embodiments, the nanoparticles have an average diameter of from about 10 nm to about 140 nm after reconstitution. In some embodiments, the nanoparticles have an average diameter of from about 10 nm to about 130 nm after reconstitution. In some embodiments, the nanoparticles have an average diameter of from about 10 nm to about 120 nm after reconstitution. In some embodiments, the nanoparticles have an average diameter of from about 10 nm to about 110 nm after reconstitution. In some embodiments, the nanoparticles have an average diameter of from about 10 nm to about 100 nm after reconstitution. In some embodiments, the nanoparticles have an average diameter of from about 10 nm to about 90 nm after reconstitution. In some embodiments, the nanoparticles have an average diameter of from about 10 nm to about 80 nm after reconstitution. In some embodiments, the nanoparticles have an average diameter of from about 10 nm to about 70 nm after reconstitution. In some embodiments, the nanoparticles have an average diameter of from about 10 nm to about 60 nm after reconstitution. In some embodiments, the nanoparticles have an average diameter of from about 10 nm to about 50 nm after reconstitution. In some embodiments, the nanoparticles have an average diameter of from about 10 nm to about 40 nm after reconstitution. In some embodiments, the nanoparticles have an average diameter of from about 10 nm to about 30 nm after reconstitution. In some embodiments, the nanoparticles have an average diameter of from about 10 nm to about 20 nm after reconstitution.

In some embodiments, the nanoparticles have an average diameter of from about 20 nm to about 1000 nm after reconstitution. In some embodiments, the nanoparticles have an average diameter of from about 20 nm to about 950 nm after reconstitution. In some embodiments, the nanoparticles have an average diameter of from about 20 nm to about 900 nm after reconstitution. In some embodiments, the nanoparticles have an average diameter of from about 20 nm to about 850 nm after reconstitution. In some embodiments, the nanoparticles have an average diameter of from about 20 nm to about 800 nm after reconstitution. In some embodiments, the nanoparticles have an average diameter of from about 20 nm to about 750 nm after reconstitution. In some embodiments, the nanoparticles have an average diameter of from about 20 nm to about 700 nm after reconstitution. In some embodiments, the nanoparticles have an average diameter of from about 20 nm to about 650 nm after reconstitution. In some embodiments, the nanoparticles have an average diameter of from about 20 nm to about 600 nm after reconstitution. In some embodiments, the nanoparticles have an average diameter of from about 20 nm to about 550 nm after reconstitution. In some embodiments, the nanoparticles have an average diameter of from about 20 nm to about 500 nm after reconstitution. In some embodiments, the nanoparticles have an average diameter of from about 20 nm to about 450 nm after reconstitution. In some embodiments, the nanoparticles have an average diameter of from about 20 nm to about 400 nm after reconstitution. In some embodiments, the nanoparticles have an average diameter of from about 20 nm to about 350 nm after reconstitution. In some embodiments, the nanoparticles have an average diameter of from about 20 nm to about 300 nm after reconstitution. In some embodiments, the nanoparticles have an average diameter of from about 20 nm to about 250 nm after reconstitution. In some embodiments, the nanoparticles have an average diameter of from about 20 nm to about 240 nm after reconstitution. In some embodiments, the nanoparticles have an average diameter of from about 20 nm to about 230 nm after reconstitution. In some embodiments, the nanoparticles have an average diameter of from about 20 nm to about 220 nm after reconstitution. In some embodiments, the nanoparticles have an average diameter of from about 20 nm to about 210 nm after reconstitution. In some embodiments, the nanoparticles have an average diameter of from about 20 nm to about 200 nm after reconstitution. In some embodiments, the nanoparticles have an average diameter of from about 20 nm to about 190 nm after reconstitution. In some embodiments, the nanoparticles have an average diameter of from about 20 nm to about 180 nm after reconstitution. In some embodiments, the nanoparticles have an average diameter of from about 20 nm to about 170 nm after reconstitution. In some embodiments, the nanoparticles have an average diameter of from about 20 nm to about 160 nm after reconstitution. In some embodiments, the nanoparticles have an average diameter of from about 20 nm to about 150 nm after reconstitution. In some embodiments, the nanoparticles have an average diameter of from about 20 nm to about 140 nm after reconstitution. In some embodiments, the nanoparticles have an average diameter of from about 20 nm to about 130 nm after reconstitution. In some embodiments, the nanoparticles have an average diameter of from about 20 nm to about 120 nm after reconstitution. In some embodiments, the nanoparticles have an average diameter of from about 20 nm to about 110 nm after reconstitution. In some embodiments, the nanoparticles have an average diameter of from about 20 nm to about 100 nm after reconstitution. In some embodiments, the nanoparticles have an average diameter of from about 20 nm to about 90 nm after reconstitution. In some embodiments, the nanoparticles have an average diameter of from about 20 nm to about 80 nm after reconstitution. In some embodiments, the nanoparticles have an average diameter of from about 20 nm to about 70 nm after reconstitution. In some embodiments, the nanoparticles have an average diameter of from about 20 nm to about 60 nm after reconstitution. In some embodiments, the nanoparticles have an average diameter of from about 20 nm to about 50 nm after reconstitution. In some embodiments, the nanoparticles have an average diameter of from about 20 nm to about 40 nm after reconstitution. In some embodiments, the nanoparticles have an average diameter of from about 20 nm to about 30 nm after reconstitution.

In some embodiments, the nanoparticles have an average diameter of from about 30 nm to about 1000 nm after reconstitution. In some embodiments, the nanoparticles have an average diameter of from about 30 nm to about 950 nm after reconstitution. In some embodiments, the nanoparticles have an average diameter of from about 30 nm to about 900 nm after reconstitution. In some embodiments, the nanoparticles have an average diameter of from about 30 nm to about 850 nm after reconstitution. In some embodiments, the nanoparticles have an average diameter of from about 30 nm to about 800 nm after reconstitution. In some embodiments, the nanoparticles have an average diameter of from about 30 nm to about 750 nm after reconstitution. In some embodiments, the nanoparticles have an average diameter of from about 30 nm to about 700 nm after reconstitution. In some embodiments, the nanoparticles have an average diameter of from about 30 nm to about 650 nm after reconstitution. In some embodiments, the nanoparticles have an average diameter of from about 30 nm to about 600 nm after reconstitution. In some embodiments, the nanoparticles have an average diameter of from about 30 nm to about 550 nm after reconstitution. In some embodiments, the nanoparticles have an average diameter of from about 30 nm to about 500 nm. In some embodiments, the nanoparticles have an average diameter of from about 30 nm to about 450 nm after reconstitution. In some embodiments, the nanoparticles have an average diameter of from about 30 nm to about 400 nm after reconstitution. In some embodiments, the nanoparticles have an average diameter of from about 30 nm to about 350 nm after reconstitution. In some embodiments, the nanoparticles have an average diameter of from about 30 nm to about 300 nm after reconstitution. In some embodiments, the nanoparticles have an average diameter of from about 30 nm to about 250 nm after reconstitution. In some embodiments, the nanoparticles have an average diameter of from about 30 nm to about 240 nm after reconstitution. In some embodiments, the nanoparticles have an average diameter of from about 30 nm to about 230 nm after reconstitution. In some embodiments, the nanoparticles have an average diameter of from about 30 nm to about 220 nm after reconstitution. In some embodiments, the nanoparticles have an average diameter of from about 30 nm to about 210 nm after reconstitution. In some embodiments, the nanoparticles have an average diameter of from about 30 nm to about 200 nm after reconstitution. In some embodiments, the nanoparticles have an average diameter of from about 30 nm to about 190 nm after reconstitution. In some embodiments, the nanoparticles have an average diameter of from about 30 nm to about 180 nm after reconstitution. In some embodiments, the nanoparticles have an average diameter of from about 30 nm to about 170 nm after reconstitution. In some embodiments, the nanoparticles have an average diameter of from about 30 nm to about 160 nm after reconstitution. In some embodiments, the nanoparticles have an average diameter of from about 30 nm to about 150 nm after reconstitution. In some embodiments, the nanoparticles have an average diameter of from about 30 nm to about 140 nm after reconstitution. In some embodiments, the nanoparticles have an average diameter of from about 30 nm to about 130 nm after reconstitution. In some embodiments, the nanoparticles have an average diameter of from about 30 nm to about 120 nm after reconstitution. In some embodiments, the nanoparticles have an average diameter of from about 30 nm to about 110 nm after reconstitution. In some embodiments, the nanoparticles have an average diameter of from about 30 nm to about 100 nm after reconstitution. In some embodiments, the nanoparticles have an average diameter of from about 30 nm to about 90 nm after reconstitution. In some embodiments, the nanoparticles have an average diameter of from about 30 nm to about 80 nm after reconstitution. In some embodiments, the nanoparticles have an average diameter of from about 30 nm to about 70 nm after reconstitution. In some embodiments, the nanoparticles have an average diameter of from about 30 nm to about 60 nm after reconstitution. In some embodiments, the nanoparticles have an average diameter of from about 30 nm to about 50 nm after reconstitution. In some embodiments, the nanoparticles have an average diameter of from about 30 nm to about 40 nm after reconstitution.

In some embodiments, the nanoparticles have an average diameter of from about 40 nm to about 1000 nm after reconstitution. In some embodiments, the nanoparticles have an average diameter of from about 40 nm to about 950 nm after reconstitution. In some embodiments, the nanoparticles have an average diameter of from about 40 nm to about 900 nm after reconstitution. In some embodiments, the nanoparticles have an average diameter of from about 40 nm to about 850 nm after reconstitution. In some embodiments, the nanoparticles have an average diameter of from about 40 nm to about 800 nm after reconstitution. In some embodiments, the nanoparticles have an average diameter of from about 40 nm to about 750 nm after reconstitution. In some embodiments, the nanoparticles have an average diameter of from about 40 nm to about 700 nm after reconstitution. In some embodiments, the nanoparticles have an average diameter of from about 40 nm to about 650 nm after reconstitution. In some embodiments, the nanoparticles have an average diameter of from about 40 nm to about 600 nm after reconstitution. In some embodiments, the nanoparticles have an average diameter of from about 40 nm to about 550 nm after reconstitution. In some embodiments, the nanoparticles have an average diameter of from about 40 nm to about 500 nm after reconstitution. In some embodiments, the nanoparticles have an average diameter of from about 40 nm to about 450 nm after reconstitution. In some embodiments, the nanoparticles have an average diameter of from about 40 nm to about 400 nm after reconstitution. In some embodiments, the nanoparticles have an average diameter of from about 40 nm to about 350 nm after reconstitution. In some embodiments, the nanoparticles have an average diameter of from about 40 nm to about 300 nm. In some embodiments, the nanoparticles have an average diameter of from about 40 nm to about 250 nm after reconstitution. In some embodiments, the nanoparticles have an average diameter of from about 40 nm to about 240 nm after reconstitution. In some embodiments, the nanoparticles have an average diameter of from about 40 nm to about 230 nm after reconstitution. In some embodiments, the nanoparticles have an average diameter of from about 40 nm to about 220 nm after reconstitution. In some embodiments, the nanoparticles have an average diameter of from about 40 nm to about 210 nm after reconstitution. In some embodiments, the nanoparticles have an average diameter of from about 40 nm to about 200 nm after reconstitution. In some embodiments, the nanoparticles have an average diameter of from about 40 nm to about 190 nm after reconstitution. In some embodiments, the nanoparticles have an average diameter of from about 40 nm to about 180 nm after reconstitution. In some embodiments, the nanoparticles have an average diameter of from about 40 nm to about 170 nm after reconstitution. In some embodiments, the nanoparticles have an average diameter of from about 40 nm to about 160 nm after reconstitution. In some embodiments, the nanoparticles have an average diameter of from about 40 nm to about 150 nm after reconstitution. In some embodiments, the nanoparticles have an average diameter of from about 40 nm to about 140 nm after reconstitution. In some embodiments, the nanoparticles have an average diameter of from about 40 nm to about 130 nm after reconstitution. In some embodiments, the nanoparticles have an average diameter of from about 40 nm to about 120 nm after reconstitution. In some embodiments, the nanoparticles have an average diameter of from about 40 nm to about 110 nm after reconstitution. In some embodiments, the nanoparticles have an average diameter of from about 40 nm to about 100 nm after reconstitution. In some embodiments, the nanoparticles have an average diameter of from about 40 nm to about 90 nm after reconstitution. In some embodiments, the nanoparticles have an average diameter of from about 40 nm to about 80 nm after reconstitution. In some embodiments, the nanoparticles have an average diameter of from about 40 nm to about 70 nm after reconstitution. In some embodiments, the nanoparticles have an average diameter of from about 40 nm to about 60 nm after reconstitution. In some embodiments, the nanoparticles have an average diameter of from about 40 nm to about 50 nm after reconstitution.

In some embodiments, the nanoparticles have an average diameter of from about 50 nm to about 1000 nm after reconstitution. In some embodiments, the nanoparticles have an average diameter of from about 50 nm to about 950 nm after reconstitution. In some embodiments, the nanoparticles have an average diameter of from about 50 nm to about 900 nm after reconstitution. In some embodiments, the nanoparticles have an average diameter of from about 50 nm to about 850 nm after reconstitution. In some embodiments, the nanoparticles have an average diameter of from about 50 nm to about 800 nm after reconstitution. In some embodiments, the nanoparticles have an average diameter of from about 50 nm to about 750 nm after reconstitution. In some embodiments, the nanoparticles have an average diameter of from about 50 nm to about 700 nm after reconstitution. In some embodiments, the nanoparticles have an average diameter of from about 50 nm to about 650 nm after reconstitution. In some embodiments, the nanoparticles have an average diameter of from about 50 nm to about 600 nm after reconstitution. In some embodiments, the nanoparticles have an average diameter of from about 50 nm to about 550 nm after reconstitution. In some embodiments, the nanoparticles have an average diameter of from about 50 nm to about 500 nm after reconstitution. In some embodiments, the nanoparticles have an average diameter of from about 50 nm to about 450 nm after reconstitution. In some embodiments, the nanoparticles have an average diameter of from about 50 nm to about 400 nm after reconstitution. In some embodiments, the nanoparticles have an average diameter of from about 50 nm to about 350 nm after reconstitution. In some embodiments, the nanoparticles have an average diameter of from about 50 nm to about 300 nm after reconstitution. In some embodiments, the nanoparticles have an average diameter of from about 50 nm to about 250 nm after reconstitution. In some embodiments, the nanoparticles have an average diameter of from about 50 nm to about 240 nm after reconstitution. In some embodiments, the nanoparticles have an average diameter of from about 50 nm to about 230 nm after reconstitution. In some embodiments, the nanoparticles have an average diameter of from about 50 nm to about 220 nm after reconstitution. In some embodiments, the nanoparticles have an average diameter of from about 50 nm to about 210 nm after reconstitution. In some embodiments, the nanoparticles have an average diameter of from about 50 nm to about 200 nm after reconstitution. In some embodiments, the nanoparticles have an average diameter of from about 50 nm to about 190 nm after reconstitution. In some embodiments, the nanoparticles have an average diameter of from about 50 nm to about 180 nm after reconstitution. In some embodiments, the nanoparticles have an average diameter of from about 50 nm to about 170 nm after reconstitution. In some embodiments, the nanoparticles have an average diameter of from about 50 nm to about 160 nm after reconstitution. In some embodiments, the nanoparticles have an average diameter of from about 50 nm to about 150 nm after reconstitution. In some embodiments, the nanoparticles have an average diameter of from about 50 nm to about 140 nm after reconstitution. In some embodiments, the nanoparticles have an average diameter of from about 50 nm to about 130 nm after reconstitution. In some embodiments, the nanoparticles have an average diameter of from about 50 nm to about 120 nm after reconstitution. In some embodiments, the nanoparticles have an average diameter of from about 50 nm to about 110 nm after reconstitution. In some embodiments, the nanoparticles have an average diameter of from about 50 nm to about 100 nm after reconstitution. In some embodiments, the nanoparticles have an average diameter of from about 50 nm to about 90 nm after reconstitution. In some embodiments, the nanoparticles have an average diameter of from about 50 nm to about 80 nm after reconstitution. In some embodiments, the nanoparticles have an average diameter of from about 50 nm to about 70 nm after reconstitution. In some embodiments, the nanoparticles have an average diameter of from about 50 nm to about 60 nm after reconstitution.

In some embodiments, the nanoparticles have an average diameter of about 10 nm after reconstitution. In some embodiments, the nanoparticles have an average diameter of about 20 nm after reconstitution. In some embodiments, the nanoparticles have an average diameter of about 30 nm after reconstitution. In some embodiments, the nanoparticles have an average diameter of about 40 nm after reconstitution. In some embodiments, the nanoparticles have an average diameter of about 50 nm after reconstitution. In some embodiments, the nanoparticles have an average diameter of about 60 nm after reconstitution. In some embodiments, the nanoparticles have an average diameter of about 70 nm after reconstitution. In some embodiments, the nanoparticles have an average diameter of about 80 nm after reconstitution. In some embodiments, the nanoparticles have an average diameter of about 90 nm after reconstitution. In some embodiments, the nanoparticles have an average diameter of about 100 nm after reconstitution. In some embodiments, the nanoparticles have an average diameter of about 110 nm after reconstitution. In some embodiments, the nanoparticles have an average diameter of about 120 nm after reconstitution. In some embodiments, the nanoparticles have an average diameter of about 130 nm after reconstitution. In some embodiments, the nanoparticles have an average diameter of about 140 nm after reconstitution. In some embodiments, the nanoparticles have an average diameter of about 150 nm after reconstitution. In some embodiments, the nanoparticles have an average diameter of about 160 nm after reconstitution. In some embodiments, the nanoparticles have an average diameter of about 170 nm after reconstitution. In some embodiments, the nanoparticles have an average diameter of about 180 nm. In some embodiments, the nanoparticles have an average diameter of about 190 nm after reconstitution. In some embodiments, the nanoparticles have an average diameter of about 200 nm after reconstitution. In some embodiments, the nanoparticles have an average diameter of about 210 nm after reconstitution. In some embodiments, the nanoparticles have an average diameter of about 220 nm after reconstitution. In some embodiments, the nanoparticles have an average diameter of about 230 nm after reconstitution. In some embodiments, the nanoparticles have an average diameter of about 240 nm after reconstitution. In some embodiments, the nanoparticles have an average diameter of about 250 nm after reconstitution. In some embodiments, the nanoparticles have an average diameter of about 300 nm after reconstitution. In some embodiments, the nanoparticles have an average diameter of about 350 nm after reconstitution. In some embodiments, the nanoparticles have an average diameter of about 400 nm after reconstitution. In some embodiments, the nanoparticles have an average diameter of about 450 nm after reconstitution. In some embodiments, the nanoparticles have an average diameter of about 500 nm after reconstitution. In some embodiments, the nanoparticles have an average diameter of about 550 nm after reconstitution. In some embodiments, the nanoparticles have an average diameter of about 600 nm after reconstitution. In some embodiments, the nanoparticles have an average diameter of about 650 nm after reconstitution. In some embodiments, the nanoparticles have an average diameter of about 700 nm after reconstitution. In some embodiments, the nanoparticles have an average diameter of about 750 nm after reconstitution. In some embodiments, the nanoparticles have an average diameter of about 800 nm. In some embodiments, the nanoparticles have an average diameter of about 850 nm after reconstitution. In some embodiments, the nanoparticles have an average diameter of about 900 nm after reconstitution. In some embodiments, the nanoparticles have an average diameter of about 950 nm after reconstitution. In some embodiments, the nanoparticles have an average diameter of about 1000 nm after reconstitution.

### Preparation of Nanoparticles

Provided in another aspect is a process of preparing any one of the compositions comprising the nanoparticles described herein, comprising:
a) dissolving a compound that is a modulator of a Bcl-2 family protein in a volatile solvent to form a solution comprising a dissolved compound that is a modulator of a Bcl-2 family protein;
b) adding the solution comprising the dissolved compound that is modulator of a Bcl-2 family protein to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form any one of the compositions described herein.

In some embodiments, the adding the solution comprising the dissolved compound that is modulator of a Bcl-2 family protein to a pharmaceutically acceptable carrier in an aqueous solution from step b) further comprises mixing to form an emulsion. In some embodiments, the mixing is performed with a homogenizer. In some embodiments, the volatile solvent is a chlorinated solvent, alcohol, ketone, ester, ether, acetonitrile, or any combination thereof. In some embodiments, volatile solvent is a chlorinated solvent. Examples of chlorinated solvents include, but are not limited to, chloroform, dichloromethane, and 1,2, dichloroethane. In some embodiments, volatile solvent is an alcohol. Examples of alcohols, include but are not limited to, methanol, ethanol, butanol (such as t-butyl and n-butyl alcohol), and propanol (such as iso-propyl alcohol). In some embodiments, volatile solvent is a ketone. An example of a ketone includes, but is not limited to, acetone. In some embodiments, volatile solvent is an ester. An example of an ester includes, but is not limited to ethyl acetate. In some embodiments, volatile solvent is an ether. In some embodiments, the volatile solvent is acetonitrile. In some embodiments, the volatile solvent is mixture of a chlorinated solvent with an alcohol.

In some embodiments, the volatile solvent is chloroform, ethanol, butanol, methanol, propanol, or a combination thereof. In some embodiments, volatile solvent is a mixture of chloroform and ethanol. In some embodiments, the volatile solvent is methanol. In some embodiments, the volatile solvent is a mixture of chloroform and methanol. In some embodiments, the volatile solvent is butanol, such as t-butanol or n-butanol. In some embodiments, the volatile solvent is a mixture of chloroform and butanol. In some embodiments, the volatile solvent is acetone. In some embodiments, the volatile solvent is acetonitrile. In some embodiments, the volatile solvent is dichloromethane. In some embodiments, the volatile solvent is 1,2 dichloroethane. In some embodiments the volatile solvent is ethyl acetate. In some embodiments, the volatile solvent is isopropyl alcohol. In some embodiments, the volatile solvent is chloroform. In some embodiments, the volatile solvent is ethanol. In some embodiments, the volatile solvent is a combination of ethanol and chloroform.

In some embodiments, the homogenization is high pressure homogenization. In some embodiments, the emulsion is cycled through high pressure homogenization for an appropriate amount of cycles. In some embodiments, the appropriate amount of cycles is from about 2 to about 10 cycles. In some embodiments, the appropriate amount of cycles is about 1, about 2, about 3, about 4, about 5, about 6, about 7, about 8, about 9, or about 10 cycles.

In some embodiments, the evaporation is accomplished with suitable equipment known for this purpose. Such suitable equipment include, but not limited to, rotary evaporators, falling film evaporators, wiped film evaporators, spray driers, and the like that can be operated in batch mode or in continuous operation. In some embodiments, the evaporation is accomplished with a rotary evaporator. In some embodiments, the evaporation is under reduced pressure.

### Administration

In some embodiments, the composition is suitable for injection. In some embodiments, the composition is suitable for parenteral administration. Examples of parenteral administration include but are not limited to subcutaneous injections, intravenous, or intramuscular injections or infusion techniques. In some embodiments, the composition is suitable for intravenous administration.

In some embodiments, the composition is administered intraperitoneally, intraarterially, intrapulmonarily, orally, by inhalation, intravesicularly, intramuscularly, intratracheally, subcutaneously, intraocularly, intrathecally, intratumorally, or transdermally. In some embodiments, the composition is administered intravenously. In some embodiments, the composition is administered intraarterially. In some embodiments, the composition is administered intrapulmonarily. In some embodiments, the composition is administered orally. In some embodiments, the composition is administered by inhalation. In some embodiments, the composition is administered intravesicularly. In some embodiments, the composition is administered intramuscularly. In some embodiments, the composition is administered intratracheally. In some embodiments, the composition is administered subcutaneously. In some embodiments, the composition is administered intraocularly. In some embodiments, the composition is administered intrathecally. In some embodiments, the composition is administered transdermally.

### Methods

Also provided herein in another aspect is a method of treating a disease in a subject in need thereof comprising administering any one of the compositions described herein.

In some embodiments, disease is cancer. Examples of cancers, include but not limited to solid tumors (e.g., tumors of the lung, breast, colon, prostate, bladder, rectum, brain or endometrium), hematological malignancies (e.g., leukemias, lymphomas, myelomas), carcinomas (e.g. bladder carcinoma, renal carcinoma, breast carcinoma, colorectal carcinoma), neuroblastoma, or melanoma. Non-limiting examples of these cancers include cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, small lymphocytic lymphoma, multiple myeloma, mesothelioma, childhood solid tumors such as brain neuroblastoma, retinoblastoma, Wilms' tumor, bone cancer and soft-tissue sarcomas, common solid tumors of adults such as head and neck cancers (e.g., oral, laryngeal and esophageal), genito urinary cancers (e.g., prostate, bladder, renal, uterine, ovarian, testicular, rectal and colon), lung cancer, breast cancer, pancreatic cancer, melanoma and other skin cancers, stomach cancer, brain cancer, liver cancer, adrenal cancer, kidney cancer, thyroid cancer, basal cell carcinoma, squamous cell carcinoma of both ulcerating and papillary type, metastatic skin carcinoma, medullary carcinoma, osteo sarcoma, Ewing's sarcoma, veticulum cell sarcoma, Kaposi's sarcoma, neuroblastoma and retinoblastoma. In some embodiments, the cancer is breast cancer, ovarian cancer, non-small cell lung cancer, pancreatic cancer, or bladder cancer.

In some embodiments, the cancer is bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer. In some embodiments, the cancer is chronic lymphocytic leukemia.

In some embodiments, the cancer is small lymphocytic lymphoma, acute lymphocytic leukemia, or acute myeloid leukemia. In some embodiments, the cancer is small lymphocytic lymphoma. In some embodiments, the cancer is acute lymphocytic leukemia. In some embodiments, the cancer is acute myeloid leukemia.

In some embodiments, the disease is solid tumor. In some embodiments, the solid tumor is a cancer of the brain, breast, cervix, colon, kidney, larynx, lung, ovary, pancreas, prostate, rectum, skin, spine, stomach, or uterus. In some embodiments, the solid tumor is a soft tissue sarcoma.

Also disclosed herein is a method of delivering a compound that is a modulator of a Bcl-2 family protein to a subject in need thereof comprising administering any one of the compositions described herein.

Disclosed compositions are administered to patients (animals and humans) in need of such treatment in dosages that will provide optimal pharmaceutical efficacy. It will be appreciated that the dose required for use in any particular application will vary from patient to patient, not only with the particular composition selected, but also with the route of administration, the nature of the condition being treated, the age and condition of the patient, concurrent medication or special diets then being followed by the patient, and other factors, with the appropriate dosage ultimately being at the discretion of the attendant physician. For treating diseases noted above, a contemplated composition disclosed herein is administered orally, subcutaneously, topically, parenterally, by inhalation spray, or rectally in dosage unit formulations containing conventional non-toxic pharmaceutically acceptable carriers, adjuvants and vehicles. Parenteral administration include subcutaneous injections, intravenous, or intramuscular injections or infusion techniques.

The following examples are provided merely as illustrative of various embodiments and shall not be construed to limit the invention in any way.

### EXAMPLES

### Exemplary Nanoparticle Compositions Containing a Modulator of a Bcl-2 Family Protein

### Example 1

This example demonstrates the preparation of a nanoparticle pharmaceutical composition comprising Compound 1 and albumin. 39.2 mL of a human albumin solution (1.47% w/v) was prepared diluting from a 25% human albumin U.S.P. solution using chloroform saturated water. Compound 1 (60 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 4 minutes. The suspension was then sterile filtered, and the average particle size (Zₐᵥ, Malvern Nano-S) was determined to be 77 nm initially, 77 nm after 15 minutes, and 78 nm after 3 days at room temperature.

### Example 2

This example demonstrates the preparation of a nanoparticle pharmaceutical composition comprising Compound 1 and albumin. 39.2 mL of a human albumin solution (1.47% w/v) was prepared diluting from a 25% human albumin U.S.P. solution using chloroform saturated water. Compound 1 (37 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 68 nm initially, and 69 nm after 2 hours at room temperature.

### Example 3

This example demonstrates the preparation of a nanoparticle pharmaceutical composition comprising Compound 1 and albumin. 19.6 mL of a human albumin solution (1.47% w/v) was prepared diluting from a 25% human albumin U.S.P. solution using chloroform saturated water. Compound 1 (11 mg) was dissolved in 400 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 82 nm initially, 82 nm after 2.5 hours, and 86 nm after 3 days at room temperature.

### Example 4

This example demonstrates the preparation of a nanoparticle pharmaceutical composition comprising Compound 2 and albumin. 39.2 mL of a human albumin solution (1.47% w/v) was prepared diluting from a 25% human albumin U.S.P. solution using chloroform saturated water. Compound 2 (67 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 79 nm initially, 79 nm after 15 minutes, and 80 nm after 24 hours at room temperature.

### Example 5

This example demonstrates the preparation of a nanoparticle pharmaceutical composition comprising Compound 2 and albumin. 39.6 mL of a human albumin solution (1.47% w/v) was prepared diluting from a 25% human albumin U.S.P. solution using chloroform saturated water. Compound 2 (67 mg) was dissolved in 400 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 129 nm initially, 129 nm after 60 minutes, and 133 nm after 24 hours at room temperature.

### Example 6

This example demonstrates the preparation of a nanoparticle pharmaceutical composition comprising Compound 3 and albumin. 39.2 mL of a human albumin solution (1.47% w/v) was prepared diluting from a 25% human albumin U.S.P. solution using chloroform saturated water. Compound 3 (56 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Nano-S) was determined to be 72 nm initially, 73 nm after 15 minutes, 73 nm after 24 hours, and 73 nm after 5 days at room temperature.

### Example 7

This example demonstrates the preparation of a nanoparticle pharmaceutical composition comprising Compound 4 and albumin. 19.2 mL of a human albumin solution (1.47% w/v) was prepared diluting from a 25% human albumin U.S.P. solution using chloroform saturated water. Compound 4 (28 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 53 nm initially, 52 nm after 15 minutes, and 51 nm after 24 hours at room temperature.

### Example 8

This example demonstrates the preparation of a nanoparticle pharmaceutical composition comprising Compound 5 and albumin. 19.6 mL of a human albumin solution (1.47% w/v) was prepared diluting from a 25% human albumin U.S.P. solution using chloroform saturated water. Compound 5 (23 mg) was dissolved in 400 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 7 minutes. The suspension was then sterile filtered, and the average particle size (Zₐᵥ, Malvern Nano-S) was determined to be 81 nm initially, 80 nm after 15 minutes, and 81 nm after 24 hours at room temperature.

### Example 9

This example demonstrates the preparation of a nanoparticle pharmaceutical composition comprising Compound 6 and albumin. 14.7 mL of a human albumin solution (1.47% w/v) was prepared diluting from a 25% human albumin U.S.P. solution using chloroform saturated water. Compound 6 (15.8 mg) was dissolved in 300 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 5 minutes. The suspension was then sterile filtered, and the average particle size (Zₐᵥ, Malvern Nano-S) was determined to be 71 nm initially, 70 nm after 15 minutes, and 70 nm after 25 hours at room temperature.

### Example 10

This example demonstrates the preparation of a nanoparticle pharmaceutical composition comprising Compound 7 and albumin. 19.6 mL of a human albumin solution (1.47% w/v) was prepared diluting from a 25% human albumin U.S.P. solution using chloroform saturated water. Compound 7 (23 mg) was dissolved in 400 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 2 minutes at high pressure (12,000 psi to 20,000 psi) while cooling (4° to 8° C.). The resulting emulsion was transferred into a rotary evaporator (Buchi, Switzerland), where the volatile solvents were removed at 40° C. under reduced pressure (approximately 25 mm Hg) for 6 minutes. The suspension was then sterile filtered, and the average particle size (Zₐᵥ, Malvern Zetasizer Nano-ZSP) was determined to be 101 nm initially, 100 nm after 60 minutes, 100 nm after 240 minutes and 101 nm after 48 hours at room temperature.

### Example 11

This example demonstrates the preparation of a nanoparticle pharmaceutical composition comprising Compound 8 and albumin. 39.2 mL of a human albumin solution (1.47% w/v) was prepared diluting from a 25% human albumin U.S.P. solution using chloroform saturated water. Compound 8 (67 mg) was dissolved in 800 μL chloroform/ethanol (90:10 ratio). The organic solvent solution was added dropwise to the albumin solution while homogenizing for 5 minutes at 5000 rpm (IKA Ultra-Turrax T 18 rotor-stator, S 18N-19G dispersing element) to form a rough emulsion. This rough emulsion was transferred into a high-pressure homogenizer (Avestin, Emulsiflex-C5), where emulsification was performed by recycling the emulsion for 3 minutes at high pressure (12,000 psi to 20,000 psi) while cooling (4° to 10° C.). The resulting emulsion was transferred into a rotary evaporator (Buchi, Switzerland), where the volatile solvents were removed at 40° C. under reduced pressure (approximately 25 mm Hg) for 8 minutes. The suspension was then sterile filtered, and the average particle size (Zₐᵥ, Malvern Nano-S) was determined to be 94 nm initially, 93 nm after 15 minutes, 93 nm after 120 minutes, and 95 nm after 24 hours at room temperature.

### Exemplary Nanoparticle Compositions Upon Lyophilization and Rehydration

### Example 12

This example demonstrates the lyophilization and rehydration into each of: water, 5% dextrose water, and saline for a nanoparticle pharmaceutical composition comprising Compound 2 and albumin. Immediately after sterile filtration, the nanoparticle suspension from Example 4 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 76 nm initially, 77 nm after 30 minutes, and 76 nm after 2 hours at room temperature. Upon hydration into 5% dextrose water, the average particle size (Zₐᵥ, Malvern Nano-S) was determined to be 88 nm initially, 87 nm after 30 minutes, and 86 nm after 2 hours at room temperature. Upon hydration into 0.9% saline, the average particle size (Zₐᵥ, Malvern Nano-S) was determined to be 75 nm initially, 73 nm after 30 minutes, and 74 nm after 2 hours at room temperature.

### Example 13

This example demonstrates the lyophilization and rehydration into each of: water, 5% dextrose water, and saline for a nanoparticle pharmaceutical composition comprising Compound 6 and albumin. Immediately after sterile filtration, the nanoparticle suspension from Example 9 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 65 nm at 20 minutes, and 101 nm after 2 hours at room temperature. Upon hydration into 5% dextrose water, the average particle size (Zₐᵥ, Malvern Nano-S) was determined to be 84 nm after 20 minutes, and 75 nm after 90 minutes, and 75 nm after 20 hours at room temperature. Upon hydration into 0.9% saline, the average particle size (Zₐᵥ, Malvern Nano-S) was determined to be 91 nm after 20 minutes, and 109 nm after 90 minutes at room temperature.

### Example 14

This example demonstrates the lyophilization and rehydration into each of: water, 5% dextrose water, and saline for a nanoparticle pharmaceutical composition comprising Compound 7 and albumin. Immediately after sterile filtration, the nanoparticle suspension from Example 10 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 99 nm initially, 99 nm after 120 minutes, and 96 nm after 24 hours at room temperature. Upon hydration into 5% dextrose water, the average particle size (Zₐᵥ, Malvern Nano-S) was determined to be 109 nm initially, 110 nm after 120 minutes, and 110 nm after 24 hours at room temperature. Upon hydration into 0.9% saline, the average particle size (Zₐᵥ, Malvern Nano-S) was determined to be 98 nm initially, 97 nm after 120 minutes, and 99 nm after 24 hours at room temperature.

### Example 15

This example demonstrates the lyophilization and rehydration into each of: water, 5% dextrose water, and saline for a nanoparticle pharmaceutical composition comprising Compound 8 and albumin. Immediately after sterile filtration, the nanoparticle suspension from Example 11 was flash frozen using a slurry of isopropyl alcohol and dry ice, followed by complete lyophilization overnight to yield a dry cake, and stored at −20° C. The cake was then reconstituted. Upon hydration into water, the average particle size (Zₐᵥ, Malvern Nano-S) was determined to be 94 nm initially, 93 nm after 30 min, and 91 nm after 2 hours at room temperature. Upon hydration into 5% dextrose water, the average particle size (Zₐᵥ, Malvern Nano-S) was determined to be 107 nm initially, 106 nm after 30 minutes, and 105 nm after 2 hours at room temperature. Upon hydration into 0.9% saline, the average particle size (Zₐᵥ, Malvern Nano-S) was determined to be 91 nm initially, 91 nm after 30 minutes, and 91 nm after 2 hours at room temperature.

What is claimed is:

1. A composition comprising nanoparticles, wherein the nanoparticles comprise a modulator of a Bcl-2 family protein; and a pharmaceutically acceptable carrier; wherein the pharmaceutically acceptable carrier comprises albumin.

2. The composition of claim 1, wherein the modulator of the Bcl-2 family protein is a modulator of Bcl-2, Bcl-xL, Bcl-w, Bcl-b, A1, and/or Mcl-1.

3. The composition of claim 1, wherein the modulator of the Bcl-2 family protein is a modulator of Bcl-2, Bcl-xL, Bcl-w, and/or Mcl-1.

4. The composition of claim 1, wherein the modulator of the Bcl-2 family protein is a modulator of Bcl-2, Bcl-xL, and/or Mcl-1.

5. The composition of any one of claims 1-4, wherein the nanoparticles have an average diameter of about 1000 nm or less for at least about 15 minutes after nanoparticle formation.

6. The composition of any one of claims 1-4, wherein the nanoparticles have an average diameter of about 10 nm or greater for at least about 15 minutes after nanoparticle formation.

7. The composition of any one of claims 1-4, the nanoparticles have an average diameter of from about 10 nm to about 1000 nm for at least about 15 minutes after nanoparticle formation.

8. The composition of any one of claims 1-4, wherein the nanoparticles have an average diameter of about 1000 nm or less for at least about 2 hours after nanoparticle formation.

9. The composition of any one of claims 1-4, wherein the nanoparticles have an average diameter of about 10 nm or greater for at least about 2 hours nanoparticle formation.

10. The composition of any one of claims 1-4, the nanoparticles have an average diameter of from about 10 nm to about 1000 nm for at least about 2 hours after nanoparticle formation.

11. The composition of any one of claims 1-10, wherein the nanoparticles have an average diameter of from about 10 nm to about 1000 nm.

12. The composition of claim 11, wherein the nanoparticles have an average diameter of from about 30 nm to about 250 nm.

13. The composition of claim 12, wherein the nanoparticles have an average diameter of from about 50 nm to about 200 nm.

14. The composition of claim 13, wherein the nanoparticles have an average diameter of from about 50 nm to about 150 nm.

15. The composition of claim 14, wherein the nanoparticles have an average diameter of from about 50 nm to about 100 nm.

16. The composition of any one of claims 1-15, wherein the albumin is human serum albumin.

17. The composition of any one of claims 1-16, wherein the molar ratio of the modulator to pharmaceutically acceptable carrier is from about 1:1 to about 20:1.

18. The composition of claim 17, wherein the molar ratio of the modulator to pharmaceutically acceptable carrier is from about 2:1 to about 12:1.

19. The composition of any one of claims 1-18, wherein the nanoparticles are suspended, dissolved, or emulsified in a liquid.

20. The composition of any one of claims 1-19, wherein the composition is sterile filterable.

21. The composition of any one of claims 1-20, wherein the composition is dehydrated.

22. The composition of claim 21, wherein the composition is a lyophilized composition.

23. The composition of claim 21 or 22, wherein the composition comprises from about 0.9% to about 24% by weight of the modulator.

24. The composition of claim 23, wherein the composition comprises from about 1.8% to about 16% by weight of the modulator.

25. The composition of any one of claims 21-24, wherein the composition comprises from about 76% to about 99% by weight of the pharmaceutically acceptable carrier.

26. The composition of claim 25, wherein the composition comprises from about 84% to about 98% by weight of the pharmaceutically acceptable carrier.

27. The composition of any one of claims 21-26, wherein the composition is reconstituted with an appropriate biocompatible liquid to provide a reconstituted composition.

28. The composition of claim 27, wherein the appropriate biocompatible liquid is a buffered solution.

29. The composition of claim 27, wherein the appropriate biocompatible liquid is a solution comprising dextrose.

30. The composition of claim 27, wherein the appropriate biocompatible liquid is a solution comprising one or more salts.

31. The composition of claim 27, wherein the appropriate biocompatible liquid is sterile water, saline, phosphate-buffered saline, 5% dextrose in water solution, Ringer's solution, or Ringer's lactate solution.

32. The composition of any one of claims 27-31, wherein the nanoparticles have an average diameter of from about 10 nm to about 1000 nm after reconstitution.

33. The composition of claim 32, wherein the nanoparticles have an average diameter of from about 30 nm to about 250 nm after reconstitution.

34. The composition of claim 33, wherein the nanoparticles have an average diameter of from about 50 nm to about 200 nm after reconstitution.

35. The composition of claim 34, wherein the nanoparticles have an average diameter of from about 50 nm to about 150 nm after reconstitution.

36. The composition of claim 35, wherein the nanoparticles have an average diameter of from about 50 nm to about 100 nm after reconstitution.

37. The composition of any one of claims 1-36, wherein the modulator is a modulator of Bcl-2.

38. The composition of any one of claims 1-36, wherein the modulator is a modulator of Bcl-xL.

39. The composition of any one of claims 1-36, wherein the modulator is a modulator of Bcl-w.

40. The composition of any one of claims 1-36, wherein the modulator is a modulator of Mcl-1.

41. The composition of any one of claims 1-36, wherein the modulator is a modulator of Bcl-2 and Bcl-xL.

42. The composition of any one of claims 1-36, wherein the modulator is a modulator of Bcl-2, Bcl-xL, and Bcl-w.

43. The composition of any one of claims 1-36, wherein the modulator is a modulator of Bcl-2, Bcl-xL, Bcl-w, and Mcl-1.

44. The composition of any one of claims 1-36, wherein the modulator is a modulator of Bcl-2, Bcl-xL, and Mcl-1.

45. The composition of claim 1, wherein the modulator is: 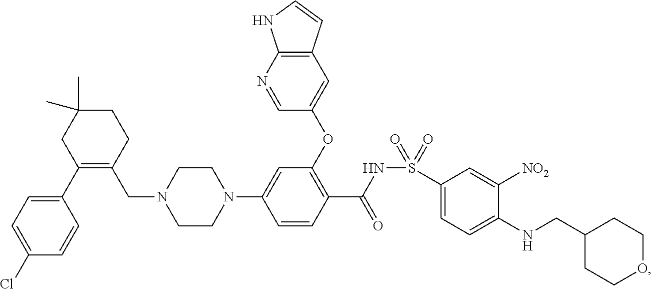 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]
or a pharmaceutically acceptable prodrug thereof.

46. The composition of claim 1, wherein the modulator is: 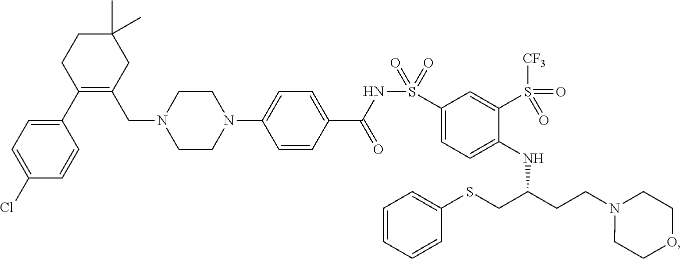 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]
or a pharmaceutically acceptable prodrug thereof.

47. The composition of claim 1, wherein the modulator is: 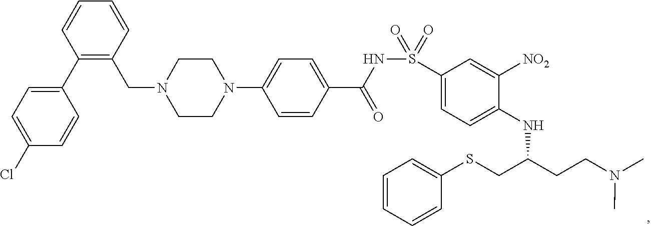 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]
or a pharmaceutically acceptable prodrug thereof.

48. The composition of claim 1, wherein the modulator is: 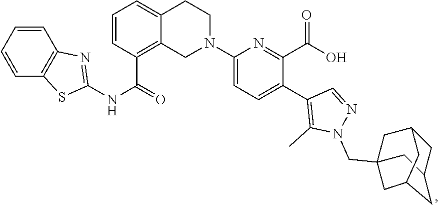 [SMILES: Cc1c(-c2ccc(N3CCc4cccc(C(=O)Nc5nc6ccccc6s5)c4C3)nc2C(=O)O)cnn1CC12CC3CC(CC(C3)C1)C2]
or a pharmaceutically acceptable prodrug thereof.

49. The composition of claim 1, wherein the modulator is: 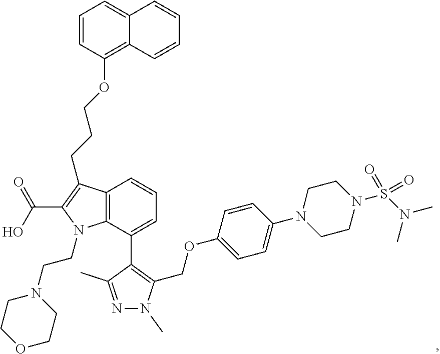 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCOCC3)c12]
or a pharmaceutically acceptable prodrug thereof.

50. The composition of claim 1, wherein the modulator is: 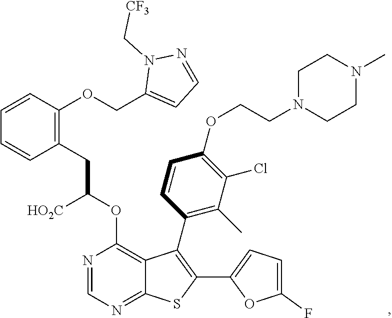
or a pharmaceutically acceptable prodrug thereof.

51. The composition of claim 1, wherein the modulator is: 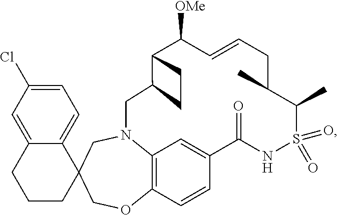 [SMILES: CO[C@H]1/C=C/C[C@H](C)[C@@H](C)S(=O)(=O)NC(=O)c2ccc3c(c2)N(C[C@@H]2CC[C@H]21)CC1(CCCc2cc(Cl)ccc21)CO3]
or a pharmaceutically acceptable prodrug thereof.

52. The composition of claim 1, wherein the modulator is: 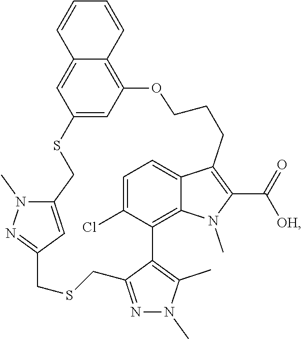 [SMILES: Cc1c2c(nn1C)CSCc1cc(n(C)n1)CSc1cc(c3ccccc3c1)OCCCc1c(C(=O)O)n(C)c3c-2c(Cl)ccc13]
or a pharmaceutically acceptable prodrug thereof.

53. The composition of claim 1, wherein the modulator is: 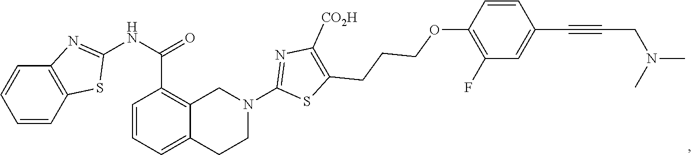 [SMILES: CN(C)CC#Cc1ccc(OCCCc2sc(N3CCc4cccc(C(=O)Nc5nc6ccccc6s5)c4C3)nc2C(=O)O)c(F)c1]
or a pharmaceutically acceptable prodrug thereof.

54. The composition of any one of claims 1-53, wherein the composition is suitable for injection.

55. The composition of any one of claims 1-54, wherein the composition is suitable for intravenous administration.

56. The composition of any one of claims 1-55, wherein the composition is administered intraperitoneally, intraarterially, intrapulmonarily, orally, by inhalation, intravesicularly, intramuscularly, intratracheally, subcutaneously, intraocularly, intrathecally, intratumorally, or transdermally.

57. A method of treating a disease in a subject in need thereof comprising administering the composition comprising nanoparticles, wherein the nanoparticles comprise a modulator of a Bcl-2 family protein; and a pharmaceutically acceptable carrier; wherein the pharmaceutically acceptable carrier comprises albumin.

58. The method of claim 57, wherein the disease is cancer.

59. The method of claim 58, wherein the cancer is bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer or spleen cancer.

60. The method of claim 59, wherein the cancer is chronic lymphocytic leukemia.

61. The method of claim 58, wherein the cancer is small lymphocytic lymphoma, acute lymphocytic leukemia, or acute myeloid leukemia.

62. The method of claim 57, wherein the disease is solid tumor.

63. The method of claim 62, wherein the solid tumor is a cancer of the brain, breast, cervix, colon, kidney, larynx, lung, ovary, pancreas, prostate, rectum, skin, spine, stomach, or uterus.

64. The method of claim 62, wherein the solid tumor is a soft tissue sarcoma.

65. A method of delivering a modulator of a Bcl-2 family protein to a subject in need thereof comprising administering the composition of any one of claims 1-56.

66. A process of preparing a composition of any one of claims 1-56 comprising
a) dissolving a modulator of a Bcl-2 family protein in a volatile solvent to form a solution comprising a dissolved modulator of a Bcl-2 family protein;
b) adding the solution comprising the dissolved modulator of a Bcl-2 family protein to a pharmaceutically acceptable carrier in an aqueous solution to form an emulsion;
c) subjecting the emulsion to homogenization to form a homogenized emulsion; and
d) subjecting the homogenized emulsion to evaporation of the volatile solvent to form the composition of any one of claims 1-56.

67. The process of claim 66, wherein the volatile solvent is a chlorinated solvent, alcohol, ketone, ester, ether, acetonitrile, or any combination thereof.

68. The process of claim 67, wherein the volatile solvent is chloroform, ethanol, methanol, or butanol.

69. The process of any one of claims 66-68, wherein the homogenization is high pressure homogenization.

70. The process of claim 69, wherein the emulsion is cycled through high pressure homogenization for an appropriate amount of cycles.

71. The process of claim 70, wherein the appropriate amount of cycles is from about 2 to about 10 cycles.

72. The process of any one of claims 66-71, wherein the evaporation is accomplished with a rotary evaporator.

73. The process of any one of claims 66-72, wherein the evaporation is under reduced pressure.
